(19) United States
(12) Patent Application Publication
(10) Pub. No.: US 2026/0146034 A1
(43) Pub. Date: May 28, 2026

(54) 1H-CYCLOPENTA[B]BENZOFURAN DERIVATIVES FOR THE PREVENTION AND TREATMENT OF VIRUS DISEASES

(71) Applicant: SJP Biotec GmbH, Meilen (CH)

(72) Inventor: Krishnaraj Rajalingam, Nieder-Olm (DE)

(21) Appl. No.: 19/110,996

(22) PCT Filed: Sep. 11, 2023
(86) PCT No.: PCT/EP2023/074929

§ 371 Date: Mar. 12, 2025

(30) Foreign Application Priority Data
Sep. 13, 2022 (EP) 22020441.6

(51) Int. Cl. C07D 307/937; A61K 31/343; A61K 31/4025; A61K 31/4525; A61K 31/496; A61K 31/5377; A61K 31/541; C07D 405/12; C07D 413/12; C07D 417/12; C07D 491/107

(52) U.S. Cl. CPC C07D 307/937; A61K 31/343; A61K 31/4025; A61K 31/4525; A61K 31/496; A61K 31/5377; A61K 31/541; C07D 405/12; C07D 413/12; C07D 417/12; C07D 491/107

(57) ABSTRACT

The present invention relates to new therapeutic uses of 1 H-Cyclopenta[b]benzofuran derivatives, specifically new uses for the treatment or prevention of a disease caused by virus infections or a disease associated with virus infections. The invention further relates to a method of treatment or prevention of diseases caused by virus infections or diseases associated with virus infections. Moreover, the invention relates to a pharmaceutical composition for use in the treatment or prevention of diseases caused by virus infections or diseases associated with virus infections.

### CROSS REFERENCE TO RELATED APPLICATIONS

This application is a U.S. National Stage of International Application No: PCT/EP2023/074929 filed Sep. 11, 2023, which depends from and claims priority to European Patent Application No: 22020441.6 filed Sep. 13, 2022.

### SEQUENCE LISTING

The instant application contains a Sequence Listing which has been submitted in.xml format via patent Center and is hereby incorporated by reference in its entirety. Said.xml copy, created on Feb. 9, 2026, is named 2026 Feb. 9 Sequence Listing SJPB0004PA.xml and is 10,051 bytes in size.

### FIELD

The present invention relates to new therapeutic uses of 1H-Cyclopenta[b]benzofuran derivatives, specifically new uses for the treatment or prevention of a disease caused by virus infections or a disease associated with virus infections. The invention further relates to a method of treatment or prevention of diseases caused by virus infections or diseases associated with virus infections. Moreover, the invention relates to a pharmaceutical composition for use in the treatment or prevention of diseases caused by virus infections or diseases associated with virus infections.

### BACKGROUND OF THE INVENTION

Virus infections pose a major challenge for public health systems throughout the world. Despite rapid scientific progress in the field of virology, the lack of effective therapeutic options and vaccines for most viral infectious diseases continues to take its toll. Emerging viral diseases pose a major threat primarily because of their rapid transmission rates and the inherent difficulties associated with developing effective vaccines and treatment options against such novel pathogens in a short span of time. Hence there is a need to develop broad-spectrum antiviral compounds that are effective against multiple viruses. Majority of currently available antivirals target one or more components directly associated with the virus life cycle. Hence the activity tends to be limited to closely related viruses, often within the same virus family. Selected nucleoside analogues including cidofovir, favipiravir and ribavirin have shown to be effective against a broad range of viruses but pose a major risk of developing antiviral resistance mainly among RNA viruses.

Viruses are infectious organic structures that spread as virions outside cells (extracellular) by transmission, but as viruses can only replicate within a suitable host cell (intracellular). They do not themselves consist of one or more cells. All viruses contain the program for their replication and spread (some viruses also contain other auxiliary components), but have neither independent replication nor their own metabolism and are therefore dependent on the metabolism of a host cell. The viruses attach to surface molecules of the host cells and introduce their genetic material into them. This penetrates into the cell nucleus and alters the cell's own DNA. The virus body (genome and proteins) replicates, sometimes on a massive scale, in the infected cell through the existing cell organelles.

A virus particle outside cells is called a virion. Virions are particles that contain nucleic acids-either deoxyribonucleic acids (DNA) or ribonucleic acids (RNA)- and usually have an enclosing protein capsule (capsid). However, a capsule is absent in the influenza virus, for example, which has a ribonucleoprotein instead. Some virions additionally possess an envelope by a bio membrane whose lipid bilayer is interspersed with viral membrane proteins. This is referred to as the viral envelope. Virions that temporarily have a viral envelope in addition to the capsid until the replication phase begins are referred to as enveloped, and viruses without such an envelope are referred to as non-enveloped.

R. Madhugiri et al., Advances in Virus Research, Vol. 96, 2016, 127, summarizes the coronavirus RNA synthesis and discuss the structural and functional features of known cis-acting RNA elements located in the 5′- and 3′-terminal untranslated region (UTR).

Previous studies have shown that Silvesterol (CAS 697235-38-4), a flavagline derivatives acts as a potent and selective inhibitor of the RNA helicase enzyme elF4A, and has broad-spectrum antiviral activity against diseases such as Ebola and coronaviruses. Silvesterol was difficult to synthesize due to the cyclo-penta-bezofuran ring and thus several modified synthetic flavaglines have been designed and successfully employed at least for in vitro preclinical studies. A couple of studies have already shown that Silvesterol has broadspectrum antiviral activity against corona viruses, SARS and MERS virus (C.Müller et al., Antiviral research 150, 2018, 123).

Similar studies have been conducted with further elF4A inhibitors. R. Cencic et al., J. Virology, 2011, 6381 describes that the compound 4E2RCat (432499-63-3), an inhibitor of elF4E-elF4G interaction, is capable of blocking coronavirus replication.

E. Gordon et al., bioRxiv Mar. 22, 2020 relates to the searching for knowledge of the molecular details of SARS-COV-2 infection. Therefore, viral proteins in human cells has been cloned, tagged and expressed. It has been identified that the human proteins physically associated with each. Further, 66 druggable human proteins or host factors targeted by 69 existing FDA-approved drugs, drugs in clinical trials and/or preclinical compounds has been identified inter alia Zotatifin (eFT226). The compounds are evaluated efficacy in live SARS-COV-2 infection assays.

eFFECTOR's Therapeutic, Apr. 30, 2020, reports that Zotatifin, an oncology product, shows anti viral activity against SARS-COV-2.

EP2457907 relates to flavagline derivatives and their use as neuroprotective, cardioprotective and antitumoral agents. In particular EP2457907 discloses the use of the described flavagline derivatives for the treatment of neuropathy. Neuropathy may result from HIV infection. This document discloses the flavalines FL42 and FL44.

EP3639820 relates to flavagline derivatives and their use as inhibitor for KRAS oncogene activation.

However, there is still a great demand for compounds that exhibit a broad-spectrum of antiviral properties.

It is therefore an object of the present invention to provide pharmaceutically active compounds that have the capability to inhibit virus activity.

### SUMMARY OF THE INVENTION

The invention relates to a compound of formula (I)

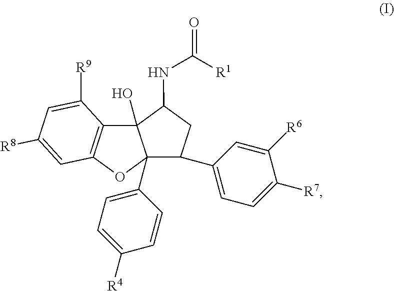

or a pharmaceutically acceptable salt thereof, wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-30 alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
for use in treatment or prevention of diseases caused by virus infections or diseases associated with virus infections.

Preferably, the present invention relates to a compound of formula (I)

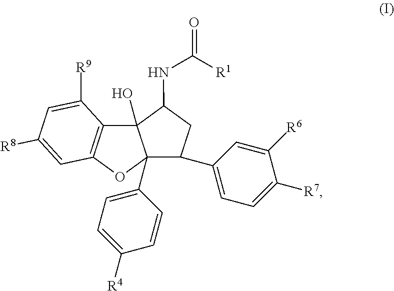

or a pharmaceutically acceptable salt thereof, wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R⁹;
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
for use in treatment or prevention of diseases caused by virus infections or diseases associated with virus infections, wherein the disease is selected from virus infection caused by positive single-stranded RNA viruses ((+) ssRNA) and negative single-stranded RNA viruses ((−) ssRNA).

In particular, the present invention relates to a compound of formula (I)

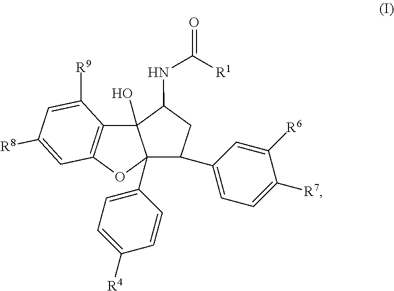

or a pharmaceutically acceptable salt thereof, wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
for use in treatment or prevention of diseases, wherein the disease is selected from virus infection caused by positive single-stranded RNA viruses ((+) ssRNA) and negative single-stranded RNA viruses ((−) ssRNA).

The invention further relates to a method of treatment or prevention of diseases caused by virus infections or diseases associated with virus infections, as defined above and below, comprising administering a therapeutically effective amount of a compound of formula (I) as defined herein or a pharmaceutically acceptable salt thereof.

Preferably, the invention further relates to a method of treatment or prevention of diseases caused by virus infections or diseases associated with virus infections, wherein the disease is selected from virus infection caused by positive single-stranded RNA viruses ((+) ssRNA) and negative single-stranded RNA viruses ((−) ssRNA), as defined above and below, as defined above and below comprising administering a therapeutically effective amount of a compound of formula (I) as defined herein or a pharmaceutically acceptable salt thereof.

In particular, the invention further relates to a method of treatment or prevention of diseases, wherein the disease is selected from virus infection caused by positive single-stranded RNA viruses ((+) ssRNA) and negative single-stranded RNA viruses ((−) ssRNA), as defined above and below, comprising administering a therapeutically effective amount of a compound of formula (I) as defined herein or a pharmaceutically acceptable salt thereof.

The invention further relates to a pharmaceutical composition comprising at least one compound of formula (I) as defined above and below, or a pharmaceutical acceptable salts thereof and pharmaceutical acceptable diluent or carrier for use in the treatment or prevention of diseases caused by virus infections or diseases associated with virus infections.

Preferably, the invention further relates to a pharmaceutical composition comprising at least one compound of formula (I) as defined above and below, or a pharmaceutical acceptable salts thereof and pharmaceutical acceptable diluent or carrier for use in the treatment or prevention of diseases caused by virus infections or diseases associated with virus infections, wherein the disease is selected from virus infection caused by positive single-stranded RNA viruses ((+) ssRNA) and negative single-stranded RNA viruses ((−) ssRNA).

In particular, the invention relates to a pharmaceutical composition comprising at least one compound of formula (I) as defined above and below, or a pharmaceutical acceptable salts thereof and pharmaceutical acceptable diluent or carrier for use in the treatment or prevention of diseases, wherein the disease is selected from virus infection caused by positive single-stranded RNA viruses ((+) ssRNA) and negative single-stranded RNA viruses ((−) ssRNA).

The invention further relates to the use of a compound of formula (I) as defined herein, to inhibit virus activity. In particular, the invention relates to the use of a compound of formula (I) as defined herein, to inhibit virus activity.

Preferably, the invention further relates to the use of a compound of formula (I) as defined herein, to inhibit virus activity. In particular, the invention relates to the use of a compound of formula (I) as defined herein, to inhibit virus activity, wherein the virus is selected from positive single-stranded RNA viruses ((+) ssRNA) and negative single-stranded RNA viruses ((−) ssRNA).

### DESCRIPTION OF THE INVENTION

The invention has the following advantages:
The compounds according to the invention exhibit advantageous virus inhibition. (+) ssRNA viruses like coronaviruses and picocorona viruses are dependent on the host 5′ Cap dependent and Cap independent strategies for viral mRNA translation initiation.
Some viruses exploit the plasma membrane associated prohibitins for their entry into host cells.
The current invention describes class of molecules that target both PHBs in the plasma membrane and elF4A complex and thereby preventing viral entry and replication.

### Compounds of Formula (I)

Unless specifically stated otherwise herein, references made in the singular may also include the plural. For example, “a” and “an” may refer to either one, or one or more.

In the context of the invention, the prefix Cₙ-Cₘ indicates the number of carbon atoms that a molecule or residue designated thereby may contain.

In the context of the invention, the expression “C₁-C₄-alkyl” refers to unbranched or branched saturated hydrocarbon groups having 1 to 4 carbon atoms. C₁-C₄-alkyl are e.g. methyl, ethyl, propyl, 1-methylethyl, butyl, 1-methylpropyl, 2-methylpropyl, 1,1-dimethylethyl.

In the context of the invention the expression “C₁-C₄-alkoxy” refers to an unbranched or branched saturated C₁-C₄-alkyl group as defined above, which is bound via an oxygen atom. Alkoxy radicals with 1 or 2 carbon atoms are preferred. C₁-C₂-alkoxy is methoxy or ethoxy. C₁-C₄-alkoxy is e.g. methoxy, ethoxy, n-propoxy, 1-methylethoxy (isopropoxy), butoxy, 1-methylpropoxy (sec-butoxy), 2-methylpropoxy (isobutoxy) or 1,1-dimethylethoxy (tert-butoxy).

In the context of the invention the expression “carbonyloxy-C₁-C₄-alkyl” refers to an unbranched or branched saturated C₁-C₄-alkyl group as defined above, which is bound via a carboxyl group.

In the context of the invention, the expressions “haloalkyl” and “haloalkoxy” refer to partially or fully halogenated alkyl or alkoxy. In other words, one or more hydrogen atoms, for example 1, 2, 3, 4 or 5 hydrogen atoms bonded to one or more carbon atoms of alkyl or alkoxy are replaced by a halogen atom, in particular by fluorine or chlorine.

In the context of the invention the expressions “hydroxyalkyl” refer to partially or fully hydroxylated alkyl. In other words, one or more hydrogen atoms, for example 1, 2, 3, 4 or 5 hydrogen atoms bonded to one or more carbon atoms of alkyl are replaced by a hydroxy atom.

In the context of the invention the expression “C₃-C₇-cycloalkyl” refers to monocyclic cycloaliphatic radicals having from 3 to 7 carbon atoms, such as cyclopropyl, cyclobutyl, cyclopentyl, cyclohexyl, cycloheptyl, cyclooctyl, cyclononyl and cyclodecyl, preferably cyclopropyl, cyclobutyl, cyclopentyl and cyclohexyl.

The expression “halogen” denotes in each case fluorine, chlorine, bromine or iodine.

In the context of the invention the expression “3-, 4-, 5-, 6- or 7-membered saturated, or partially unsaturated heterocyclic ring containing 1, 2 or 3 heteroatoms or heteroatom containing groups, wherein those heteroatom(s) (group(s)) are selected from N, O, S, NR^{c}, SO and SO₂ are ring members”, refers to monocyclic or polycyclic radicals that are attached to the remainder of the molecule via a nitrogen ring member. “Heterocyclic ring” also in particular comprises “polycyclic”, for example bicyclic, tricyclic or tetracyclic ring systems, in which one of the abovementioned monocyclic heterocylcyl residues is condensed or bridged with at least one further, identical or different heterocyclic ring or, at least one cycloalkyl according to the above definition in each case. Examples of 3-, 4-, 5-, 6- or 7-membered saturated heterocyclic rings include: aziridinyl, azetidinyl, pyrrolidinyl, pyrazolidinyl, pyrazolidinyl, imidazolidinyl, oxazolidinyl, isoxazolidinyl, thiazolidinyl, isothiazolidinyl, 1,2,4-oxadiazolidinyl, 1,2,4-thiadiazolidinyl, 1,2,4 triazolidinyl, 1,3,4-oxadiazolidinyl, 1,3,4 thiadiazolidinyl, 1,3,4 triazolidinyl, piperidinyl, hexahydropyridazinyl, hexahydropyrimidinyl, hexahydropyrimidinyl, piperazinyl, 1,3,5-hexahydrotriazinyl, 1,2,4 hexahydrotriazinyl, morpholinyl, 2-thiomorpholinyl, 3-thiomorpholinyl, 1-oxothiomorpholinyl, 1-oxothiomorpholinyl, 1,1-dioxothiomorpholinyl, 1,1-dioxothiomorpholinyl, hexahydroazepinyl, hexahydrooxepinyl, hexahydrodiazepinyl, hexahydrooxazepinyl and the like. Examples of 3-, 4-, 5-, 6- or 7-membered partially unsaturated heterocyclic rings include: pyrrolinyl, isoxazolinyl, isothiazolinyl, dihydropyrazolyl, tetrahydropyridinyl, tetrahydropyridazinyl, tetrahydropyridazinyl and tetrahydropyrimidinyl. Examples of 3-, 4-, 5-, 6- or 7-membered polycyclic ring system, in particular a bi-, tri- or tetracyclic ring system include: 8-methyl-3,8-diazabicyclo[3.2.1]octan-3-yl.

In the context of the invention the expression “C₃-C₇ heterocyloalkyl” refers to saturated cycloaliphatic groups having 3 to 7, preferably 3 to 6 ring atoms, in which 1, 2 or 3 of the ring carbon atoms have been replaced by heteroatoms or heteroatom-containing groups, preferably selected from NR^{c}, O, S, SO and SO₂ and which may be optionally substituted. In the case of a substitution, these heterocycloaliphatic groups preferably have 1, 2 or 3, particularly preferably 1 or 2, particularly 1 substituent(s). The heterocyloalkyl refers to monocyclic radicals that are attached to the remainder of the molecule via a carbon ring member. By way of example of such heterocycloaliphatic residues, mention may be made of aziridinyl, azetidinyl, pyrrolidinyl, pyrazolidinyl, imidazolidinyl, oxazolidinyl, isoxazolidinyl, thiazolidinyl, isothiazolidinyl, 1,2,4-oxadiazolidinyl, 1,2,4-thiadiazolidinyl, 1,2,4 triazolidinyl, 1,3,4-oxadiazolidinyl, 1,3,4 thiadiazolidinyl, 1,3,4 triazolidinyl, piperidinyl, hexahydropyridazinyl, hexahydropyrimidinyl, hexahydropyrimidinyl, piperazinyl, 1,3,5-hexahydrotriazinyl, 1,2,4 hexahydrotriazinyl, morpholinyl, 2-thiomorpholinyl, 3-thiomorpholinyl, 1-oxothiomorpholinyl, 1-oxothiomorpholinyl, 1,1-dioxothiomorpholinyl, 1,1-dioxothiomorpholinyl, hexahydroazepinyl, hexahydrooxepinyl, hexahydrodiazepinyl, hexahydrooxazepinyl and the like.

In the context of the invention the expression “spiro” refers to compounds, which have at least two molecular rings with only one common atom.

The compounds of formula (I), in particular of formulae (I.a), (I.b), (A), (B), (C), (D), (E), (F), (G), (H), (I), (J), (K), (L), (M), (N), (O), (P), (Q), (R), (S), (T), (U), (V), (W) an enantiomeric mixture thereof, may form salts which are also within the scope of this invention. The term “salt(s)” as employed herein, denotes acidic and/or basic salts formed with inorganic and/or organic acids and bases. Pharmaceutically acceptable (i.e. non-toxic, physiologically acceptable) salts are preferred, although other salts are also useful, e.g., in isolation or purification steps which may be employed during preparation. Salts of the compounds of formula (I), in particular of formulae (I.a), (I.b), (A), (B), (C), (D), (E), (F), (G), (H), (I), (J), (K), (L), (M), (N), (O), (P), (Q), (R), (S), (T), (U), (V), (W) or an enantiomeric mixture thereof may be formed, for example, by reacting a compound of formula (I), in particular of formulae (I.a), (I.b), (A), (B), (C), (D), (E), (F), (G), (H), (I), (J), (K), (L), (M), (N), (O), (P), (Q), (R), (S), (T), (U), (V), (W) or an enantiomeric mixture thereof with at least one acid or base. The acid or base is added in an amount suitable for partial or complete neutralization, e.g. an equivalent amount.

The phrase “pharmaceutically acceptable salt(s)” as used herein, unless otherwise indicated, includes salts containing pharmacologically acceptable anions or cations, such as chloride, bromide, iodide, nitrate, sulfate, bisulfate, phosphate, hydrogen phosphate, dihydrogen phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, sulfate, benzenesulfonate, p-toluenesulfonate and palmoate [i.e. 4,4′-methylene-bis-(3-hydroxy-2-naphthoate)] salts.

In the context of the present invention, a chemical structure that does not explicitly show a specific stereochemical orientation usually means all possible stereoisomers and mixtures thereof, unless indicated otherwise, for example,

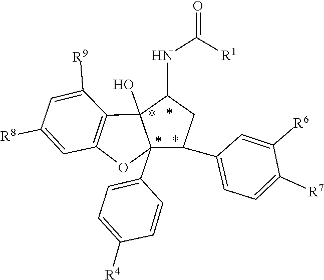

in which * designates the asymmetry centers.

“Chiral compounds” in the sense of the invention are compounds that contain no improper axis of rotation (Sₙ). In the context of the present invention, they are in particular compounds with at least four chirality centers and without Sₙ-symmetry.

“Stereoisomers” in the context of the invention are compounds of identical constitution but different atomic arrangement in the three-dimensional space.

“Enantiomers” are stereoisomers which behave like image to mirror image to one another, e.g. compounds of formulae (I.a) and (I.b) are enantiomers. The “enantiomeric excess” (ee) achieved during asymmetric synthesis is given here by the following formula: ee [%]=(R−S)/(R+S)×100. R and S are the descriptors of the CIP system for the two enantiomers and describe the absolute configuration on the asymmetric atom. The enantiomerically pure compound (ee=100%) is also referred to as “homochiral compound”.

“Diastereomers” are stereoisomers which are not enantiomeric to one another.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A “stereoisomer” refers to a compound made up of the same atoms bonded by the same bonds but having different three-dimensional structures, which are not interchangeable.

The present invention contemplates various stereoisomers and mixtures thereof and includes “enantiomers”, which refers to two stereoisomers whose molecules are nonsuperimposable mirror images of one another.

Compounds of the invention or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography.

Relative configuration in stereochemistry (relative stereochemistry) is the arrangement of atoms or groups of atoms that is described relative to other atoms or groups of atoms in the molecule. In other words, this term describes the position of atoms or groups of atoms in space in relation to other atoms or groups of atoms that are located elsewhere in the molecule.

Absolute configuration in stereochemistry (absolute stereochemistry) is the arrangement of atoms or group of atoms that is described independently of any other atom or group of atoms in the molecule. This type of configuration is defined for chiral molecular entities and their stereochemical descriptions (e.g. R or S).

Syn means that with regard to the orientation of the substituents on the 5-membered ring they are bound to (4 asymmetric carbon atoms) all substituents point in the same direction relative to the plane of the 5-membered ring.

The indication (+/−) in the formulae according to the invention indicates that the compounds are present as a racemic mixture.

Racemic mixture or racemate is defined as a mixture of compounds consisting of two molecules structured like image and mirror image (=enantiomers) and which are present in an equimolar mixture, i.e. in the ratio 1:1 (50:50).

Compounds of formula (I.a′)

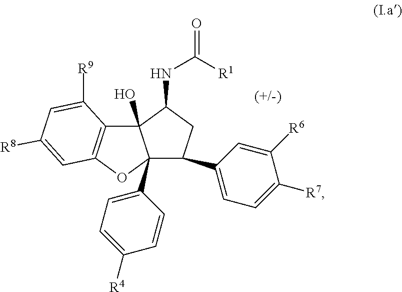

wherein R¹, R⁴, R⁶, R⁷, R⁸ and R⁹ have one of the meanings as defined above or below, relates to compounds, wherein the relative stereochemistry of the compounds is specified.

The compounds of formula (I.a′) have a relative stereochemistry of all syn and are a racemic mixture of two enantiomers (all 4 substituents bound on the 5-membered ring oriented in the same direction).

One preferred embodiment of the invention is a racemic mixture of formula (I.a′).

In other words compounds (I.a′) ((+/−)) relates to a mixture of compounds (I.a) and (I.b), which are depicted below, wherein the ratio of (I.a):(I.b) is 1:1

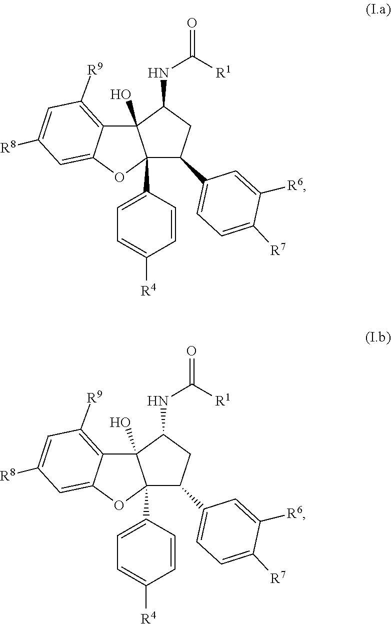

wherein R¹, R⁴, R⁶, R⁷, R⁸ and R⁹ have one of the meanings as defined above or below.

The compounds of formula (I)

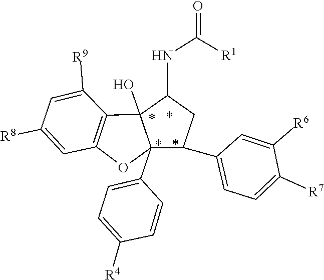

in which * designates the asymmetry centers, represent the isomers of formulas (I.a), (I.b), (I.c), (I.d), (I.e), (I.f), (I.g), (I.h), (I.i), (I.j), (I.k), (I.l), (I.m), (I.n), (I.o) and (I.p):

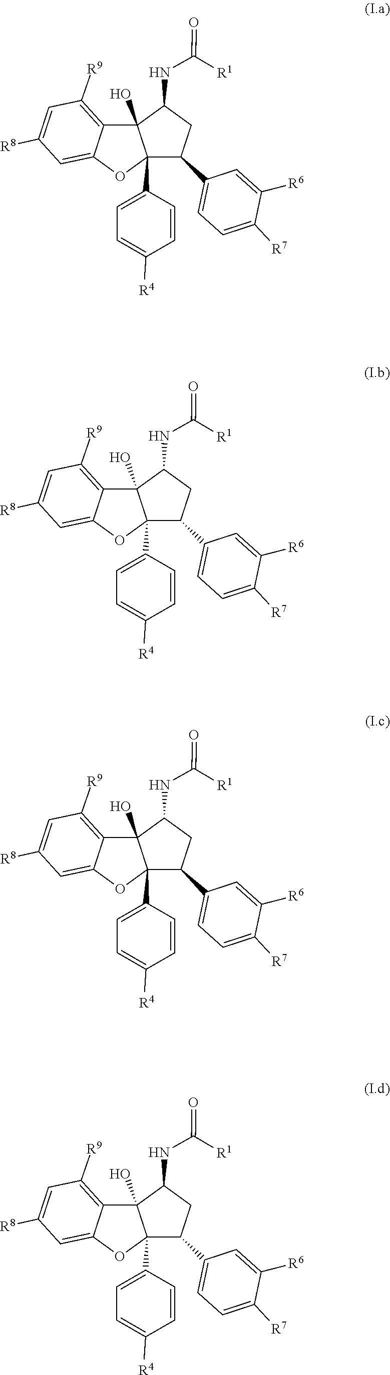

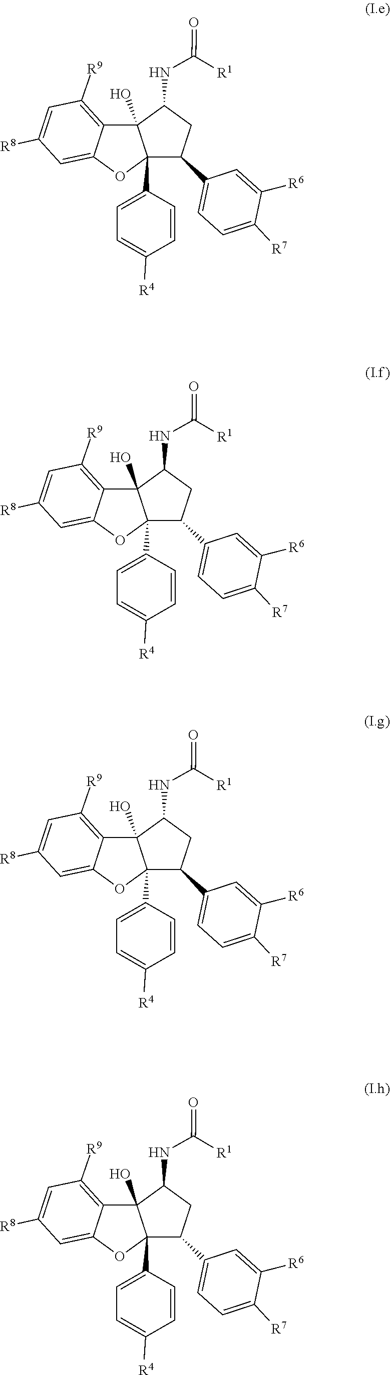

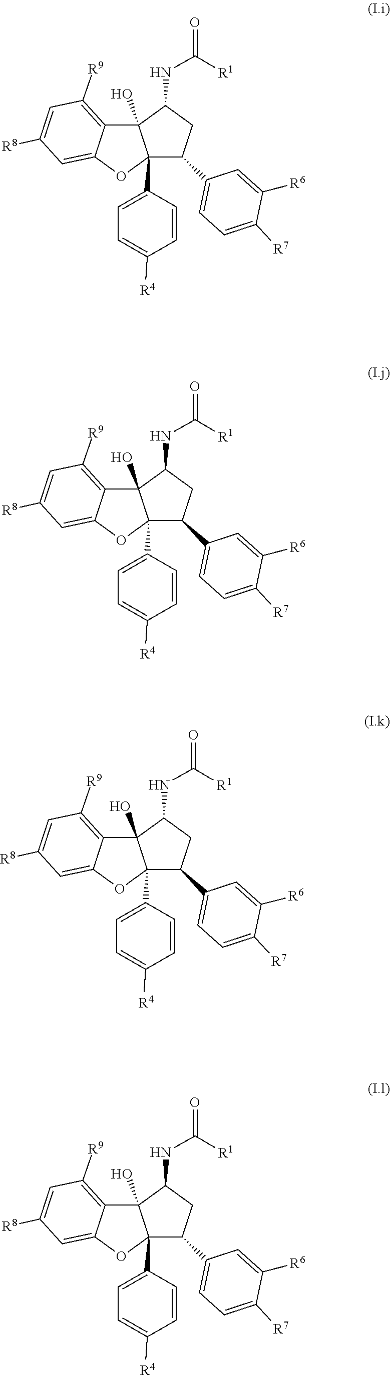

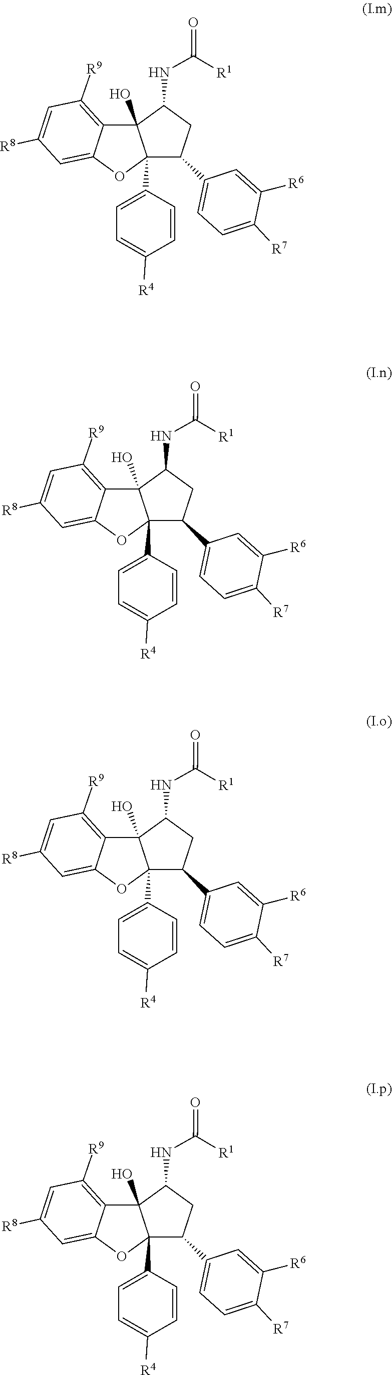

wherein, R¹, R⁴, R⁶, R⁷, R⁸ and R⁹ have one of the meanings as defined above or below.

The compounds of formulae (I.a) to (I.p) are specified by their absolute stereochemistry.

In a preferred embodiment the compound of formula (I) is a mixture of at least two enantiomers (I.a) to (I.p) or a mixture of the pharmaceutically acceptable salt thereof, wherein one enantiomer is enriched.

Preferably the compound of formula (I) is a mixture of (I.a) and (I.b) or a mixture of the pharmaceutically acceptable salt thereof, wherein the enantiomer excess (ee) of the enantiomer of formula (I.a) is at least 20%, preferably at least 50%, in particular at least 80%, especially at least 99%.

Preferred are compound of formula (I) according to the invention, or a pharmaceutically acceptable salt thereof, wherein R⁸ and R⁹ are both OCH₃.

Further, preferred are compound of formula (I) according to the invention, or a pharmaceutically acceptable salt thereof, wherein R⁶ is F and R⁷ is hydrogen.

Preferred are compounds of formula (I), wherein R¹ is selected from
C₁-C₄ alkyl, which is unsubstituted or substituted by 1, 2 or 3 substituents R^{a}; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} S, SO and SO₂, wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi-or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}; or
R² and R³ together with the nitrogen atom, which they are attached to, form a saturated or partly unsaturated spiro moiety, comprising 1, 2 identical or different heteroatoms or heteroatom-containing groups as ring members, selected from N, NR^{c} and O and wherein the heterocyclic ring is unsubstituted or substituted by 1, 2 or 3 identical or different radicals R^{g};
R⁴, R^{a}, R^{b}, R^{c},R^{d}, R^{e}, R^{f} and R⁴ have one of the meanings as defined above and below.

Preferred are compounds of formula (I), wherein R¹ is selected from
C₁-C₄ alkyl, which is unsubstituted or substituted by 1, 2 or 3 substituents R^{a};
NR²R³, wherein R² and R³ independently from each other are selected from hydrogen and C₁-C₄ alkyl, and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} or O, wherein the heterocyclic rings are unsubstituted or substituted by 1, 2, 3, 4 or 5 identical or different radicals R^{d}.

In particular R¹ is selected from
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably R² and R³ together with the nitrogen atom, which they are attached to, form a 2-oxa-6-azaspiro[3.3]heptan-6-yl spiro compound.

More preferably, R¹ is selected from
C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom, preferably C₅-C₇ heterocyloalkyl is selected from pyrrolidinyl and piperidinyl; or
NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents preferably hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{c} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl) 2 and NH(C₁-C₂ alkyl), preferably R² and R³ together with the nitrogen atom, which they are attached to, form a pyrrolidine ring, piperazine ring, acetidine ring or morpholine ring wherein the pyrrolidine ring, piperazine ring, acetidine ring, or morpholine ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) carbonyloxy-C₁-C₂-alkyl; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

Particularly, R¹ is NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents, especially R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O, wherein R^{c} is selected from hydrogen and C₁-C₄-alkyl. Preferably R² and R³ together with the nitrogen atom, which they are attached to, form a pyrrolidine ring, piperazine ring, acetidin ring or morpholin ring, wherein the pyrrolidine ring, piperazine ring, acetidin ring and morpholin ring is unsubstituted or substituted by 1 or 2 substituents selected from C₁-C₄-alkyl, C₁-C₄-hydroxyalkyl and C₁-C₄-alkoxy.

In particular, R¹ is selected from methyl, 4-methyl-piperazin-1-yl, pyrrolidin-1-yl, N,N-diethylamino, N-isopropylamino, N-ethylamino, N, N-methyl-isopropylamino, acetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1,1dioxide (1λ⁶-thiomorpholine-1,1-dionyl), 3-(dimethylamino) azetidin-1-yl, 4-(dimethylamino) piperidin-1-yl, N-ethan-1-ol-amino, azetidine-3-carbonyloxymethyl, N, N-dimethylamino methyl, 2-oxa-6-azaspiro[3.3]heptan-6-yl and pyrrolidine-3-yl especially, methyl, 4-methyl-piperazin-1-yl, pyrrolidin-1-yl, N, N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino.

In a first preferred embodiment R¹ is selected from C₁-C₄ alkyl, which is unsubstituted or substituted by 1, 2 or 3 substituents R^{a}, in particular R¹ is selected from C₁-C₂ alkyl, especially R¹ is methyl.

In a second preferred embodiment R¹ is selected from NR²R³, wherein R² and R³ independently from each other are selected from hydrogen and C₂-C₃ alkyl, which is unsubstituted or substituted by 1 or 2 substituents R^{a}, preferably C₂-C₃ alkyl, which is unsubstituted. In particular, R¹ is hydrogen and R² is C₂-C₃ alkyl, which is unsubstituted, especially, R¹ is hydrogen and R² is selected from ethyl and isopropyl.

In a third preferred embodiment R¹ is selected from NR²R³, wherein R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O. Preferably R² and R³ together with the nitrogen atom, which they are attached to, form a pyrrolidine ring, piperazine ring, acetidin ring or morpholin ring, wherein the pyrrolidine ring, piperazine ring, acetidin ring or morpholin ring is unsubstituted or substituted by 1 or 2 substituents selected from C₁-C₄-alkyl, C₁-C₄-hydroxyalkyl and C₁-C₄-alkoxy.

In particular R² and R³ together with the nitrogen atom, which they are attached to, form a piperazin-1-yl ring, N—, acetidin-1-yl or morpholin-4-yl or pyrrolidin-1-yl ring, ecpecially a 4-methyl-piperazin-1-yl ring, pyrrolidin-1-yl ring, acetidin-1-yl or morpholin-4-yl.

In a fourth preferred embodiment R¹ is selected from C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom, in particular R¹ is selected from C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom.

In a fifth preferred embodiment R¹ is selected from NR²R³, wherein R² and R³ together with the nitrogen atom, which they are attached to, form a saturated or partly unsaturated bi-or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}, in particular R¹ is selected from NR²R³, wherein R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

In a sixth preferred embodiment R¹ is selected from NR²R³, wherein R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R⁹, in particular R¹ is selected from NR²R³, wherein R² and R³ together with the nitrogen atom, which they are attached to, form a saturated or partly unsaturated spiro compound, comprising 1 or 2 identical or different heteroatoms or heteroatom-containing groups as ring members, selected from N, NR^{c} and O and wherein the heterocyclic ring is unsubstituted or substituted by 1, 2 or 3 identical or different radicals R⁹, preferably R² and R³ together with the nitrogen atom, which they are attached to, form saturated or partly unsaturated spiro moiety, comprising 2 identical or different heteroatoms or heteroatom-containing groups as ring members, selected from N, NR^{c} and O, especially R² and R³ together with the nitrogen atom, which they are attached to, form a 2-oxa-6-azaspiro[3.3]heptan-6-yl spiro compound.

In particular, R⁴ is selected from Cl, CN and C₃-C₆ cycloalkyl. Especially, R⁴ is selected from Cl, CN and cyclopropyl.

Irrespective of its occurrence, R^{a} is preferably selected from C₁-C₂-alkyl, C₃-C₆ cycloalkyl, and phenyl, wherein C₃-C₆ cycloalkyl and phenyl is unsubstituted or substituted by 1 or 2 substituents selected from F and Cl. Especially, R^{a} is selected from C₁-C₂-alkyl, C₃-C₆ cycloalkyl, and 4-fluoro-phenyl.

Irrespective of its occurrence, R^{b} is preferably selected from C₁-C₂-alkyl.

Irrespective of its occurrence, R^{c} is preferably selected from hydrogen and C₁-C₄-alkyl, in particular C₁-C₂-alkyl, especially methyl.

Irrespective of its occurrence, R^{d} is preferably selected from C₁-C₄-alkyl, C₁-C₄-hydroxyalkyl and C₁-C₄-alkoxy, in particular C₁-C₂-alkyl and C₁-C₂-hydroxyalkyl.

Irrespective of its occurrence, R^{e} is selected from halogen, C₁-C₄-alkyl, C₁-C₄-haloalkyl and NR^{5a}R^{5b}.

Irrespective of its occurrence, R′ is preferably selected from halogen, C₁-C₄-alkyl and C₁-C₄-haloalkyl.

Irrespective of its occurrence, R^{g} is preferably selected from halogen, C₁-C₄-alky and C₁-C₄-haloalkyl.

Irrespective of its occurrence, R^{h} is preferably selected from halogen, C₁-C₄-alkyl and C₁-C₄-haloalkyl.

Irrespective of its occurrence, R^{5a} and R^{5b} independently of each other are preferably selected from hydrogen and C₁-C₄-alkyl.

Another preferred embodiment are the compound of formula (I) as defined above and below or a mixture of the pharmaceutically acceptable salt thereof, which is a racemic mixture (I.a′)

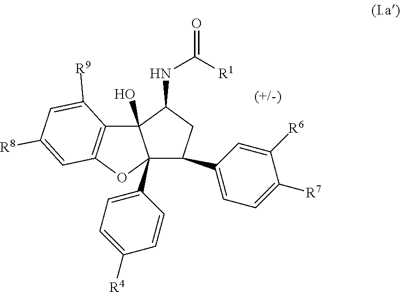

wherein R¹, R⁴, R⁶, R⁷, R⁸ and R⁹ have the meanings as defined above.

Another preferred embodiment is the compound of formula (I), which is a compound of formula (I.a) or an enantiomeric mixture comprising the compounds of formula (I.a) and (I.b) or the pharmaceutically acceptable salt thereof,

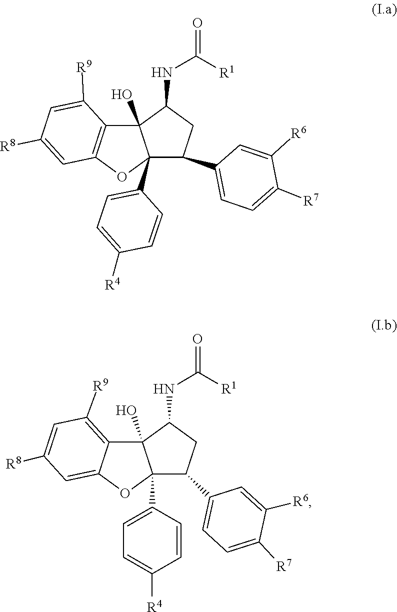

wherein R¹, R⁴, R⁶, R⁷, R⁸ and R⁹ have the same meanings as defined above and below.

In particular, the compound of formula (I) is a mixture of (I.a) and (I.b) or a mixture of the pharmaceutically acceptable salt thereof, wherein the enantiomeric excess (ee) of the enantiomer of formula (I.a) is at least 20%, preferably at least 50%, in particular at least 80%, especially at least 99%.

Especially the compound of formula (I) is a compound (I.a) a pharmaceutically acceptable salt thereof,

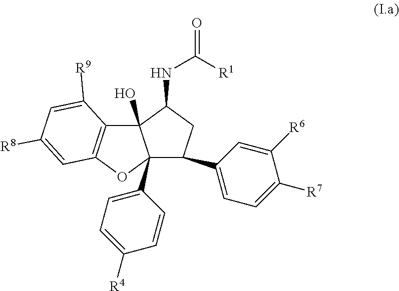

wherein R¹, R⁴, R⁶, R⁷, R⁸ and R⁹ have the meanings as defined above.

Preferred are compounds of formulae (I.a), or the racemic mixture (I.a′), or an enantiomeric mixture comprising the compounds of formula (I.a) and (I.b) in a different ratios from 1:1, or a pharmaceutically acceptable salt thereof, wherein R¹ is selected from
C₁-C₄ alkyl, wherein alkyl is unsubstituted or substituted by 1, 2 or 3 substituents R^{a}; or
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f}; or
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R⁹; or
R^{a} is selected from halogen, OH, C₃-C₇ cycloalkyl, C₁-C₃ alkoxy, phenyl, NR⁵ªR^{5b}, C₁-C₄-alkylsulfonyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR⁵ªR^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃ and OCD₃;
R⁹ is selected from OCH₃ and OCD₃.

Preferred are compounds of formula (I) according to the invention, or a pharmaceutically acceptable salt thereof, wherein R⁸ and R⁹ are both OCH₃.

Further, preferred are compounds of formula (I) according to the invention, or a pharmaceutically acceptable salt thereof, wherein R⁶ is F and R⁷ is hydrogen. Preferred are compounds of formulae (I.a), (I.a′) or a pharmaceutically acceptable salt thereof, wherein R¹ is selected from
C₁-C₄ alkyl, which is unsubstituted or substituted by 1, 2 or 3 substituents R^{a};
NR²R³, wherein R² and R³ independently from each other are selected from hydrogen and C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} and O, wherein the heterocyclic rings are unsubstituted or substituted by 1, 2, 3, 4 or 5 identical or different radicals R^{d}.

In particular R¹ is selected from
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably
R² and R³ together with the nitrogen atom, which they are attached to, form a 2-oxa-6-azaspiro[3.3]heptan-6-yl spiro compound.

More preferably, R¹ is is selected from
C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom, preferably C₅-C₇ heterocyloalkyl is selected from pyrrolidinyl and piperidinyl; or
NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents, preferably hydrogen, C₂-C₃ alkyl, which is unsubstituted, and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{c} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl)₂ and NH(C₁-C₂ alkyl), preferably R² and R³ together with the nitrogen atom, which they are attached to, form a pyrrolidine ring, piperazine ring, acetidin ring or morpholin ring wherein the pyrrolidine ring, piperazine ring, acetidin ring or morpholin ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) carbonyloxy-C₁-C₂-alkyl; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

Particularly, R¹ is NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents R^{b}, especially R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O, wherein R^{c} is selected from hydrogen and C₁-C₄-alkyl. Preferably R² and R³ together with the nitrogen atom, which they are attached to, form a pyrrolidine ring, piperazine ring, acetidin ring or morpholin ring, wherein the pyrrolidine ring, piperazine ring, acetidin ring and morpholin ring is unsubstituted or substituted by 1 or 2 substituents selected from C₁-C₄-alkyl, C₁-C₄-hydroxyalkyl and C₁-C₄-alkoxy.

In particular, R¹ is selected from methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1, 1dioxide (1λ⁶-thiomorpholine-1,1-dionyl), 3-(dimethylamino) azetidin-1-yl, 4-(dimethylamino) piperidin-1-yl, N-ethan-1-ol-amino, azetidine-3-carbonyloxymethyl, N,N-dimethylaminometkyl, 2-oxa-6-azaspiro[3.3]heptan-6-yl and pyrrolidine-3-yl especially, methyl, 4-methyl-piperazin-1-yl, pyrrolidin-1-yl, N, N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino and (cyclopropylmethyl)amino.

In a first preferred embodiment R¹ is selected from C₁-C₄ alkyl, which is unsubstituted or substituted by 1, 2 or 3 substituents R^{a}, in particular R¹ is selected from C₁-C₂ alkyl, especially R¹ is methyl.

In a second preferred embodiment R¹ is selected from NR²R³, wherein R² and R³ independently from each other are selected from hydrogen and C₂-C₃ alkyl, which is unsubstituted or substituted by 1 or 2 substituents R^{a}, preferably C₂-C₃ alkyl, which is unsubstituted. In particular, R¹ is hydrogen and R² is C₂-C₃ alkyl, which is unsubstituted, especially, R¹ is hydrogen and R² is selected from ethyl and isopropyl.

In a third preferred embodiment R¹ is selected from NR²R³, wherein R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O. Preferably R² and R³ together with the nitrogen atom, which they are attached to, form a pyrrolidine ring, piperazine ring, acetidin ring or morpholin ring, wherein the pyrrolidine ring, piperazine ring, acetidin ring or morpholin ring is unsubstituted or substituted by 1 or 2 substituents selected from C₁-C₄-alkyl, C₁-C₄-hydroxyalkyl and C₁-C₄-alkoxy. In particular R² and R³ together with the nitrogen atom, which they are attached to, form a piperazin-1-yl ring, N-acetidin-1-yl ring or morpholin-4-yl ring or pyrrolidin-1-yl ring, especially a 4-methyl-piperazin-1-yl ring, pyrrolidin-1-yl ring, acetidin-1-yl or morpholin-4-yl.

In a fourth preferred embodiment R¹ is selected from C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom, in particular R¹ is selected from C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom.

In a fifth preferred embodiment R¹ is selected NR²R³, wherein from R² and R³ together with the nitrogen atom, which they are attached to, form a saturated or partly unsaturated bi-or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}, in particular R¹ is selected NR²R³, wherein from R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}. Preferably R² and R³ together with the nitrogen atom, which they are attached to, form a 8-methyl-3,8-diazabicyclo[3.2.1]octan-3-yl ring system.

In a sixth preferred embodiment R¹ is selected from NR²R³, wherein R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g}, in particular R¹ is selected from NR²R³, wherein R² and R³ together with the nitrogen atom, which they are attached to, form a saturated or partly unsaturated spiro moiety, comprising 1, 2 identical or different heteroatoms or heteroatom-containing groups as ring members, selected from N, NR^{c} and O and wherein the heterocyclic ring is unsubstituted or substituted by 1, 2 or 3 identical or different radicals R⁹, preferably R² and R³ together with the nitrogen atom, which they are attached to, form saturated or partly unsaturated spiro compound, comprising 2 identical or different heteroatoms or heteroatom-containing groups as ring members, selected from N, NR^{c} and O, especially R² and R³ together with the nitrogen atom, which they are attached to, form a 2-oxa-6-azaspiro[3.3]heptan-6-yl spiro compound.

In particular R⁴ is selected from Cl, CN and C₃-C₆ cycloalkyl. Especially, R⁴ is selected from Cl, CN and cyclopropyl.

Another special embodiment are the compounds of formula (I-A)

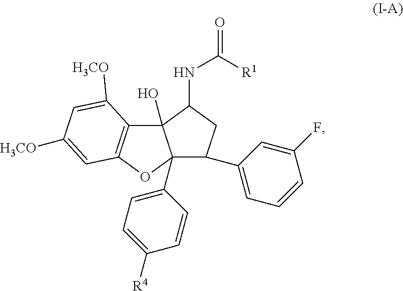

wherein R¹ and R⁴ are selected from the definition given in one line of table 1:

TABLE 1

| Nr. | R¹ | R⁴ |
|---|---|---|
| 1. | N,N-dimethylamino | Cl |
| 2. | 4-methyl-piperazin-1-yl | Cl |
| 3. | pyrrolidin-1-yl | Cl |
| 4. | N,N-diethylamino | Cl |
| 5. | N-isopropylamino | Cl |
| 6. | N-ethylamino | Cl |
| 7. | N,N-methyl-isopropylamino | Cl |
| 8. | acetidin-1-yl | Cl |
| 9. | morpholin-4-yl | Cl |
| 10. | N-cyclopentylamino | Cl |
| 11. | [1-(4-fluorophenyl)ethyl]amino | Cl |
| 12. | (cyclopropylmethyl)amino | Cl |
| 13. | methyl | Cl |
| 14. | 8-methyl-3,8- | Cl |
| | diazabicyclo[3.2.1]octan-3-yl | |
| 15. | 1-methylpiperidin-4-yl | Cl |
| 16. | thiomorpholine-4-yl-1,1dioxide | Cl |
| 17. | 3-(dimethylamino)azetidin-1-yl | Cl |
| 18. | 4-(dimethylamino)piperidin-1-yl | Cl |
| 19. | N-ethan-1-ol-amino | Cl |
| 20. | azetidine-3-carbonyloxy methyl | Cl |
| 21. | N,N-dimethylamino methyl | Cl |
| 22. | 2-oxa-6-azaspiro[3.3]heptan-6-yl | Cl |
| 23. | pyrrolidine-3-yl | Cl |
| 24. | (piperazin-1-yl)ethyl | Cl |
| 25. | N,N-dimethylamino | cyclopropyl |
| 26. | 4-methyl-piperazin-1-yl | cyclopropyl |
| 27. | pyrrolidin-1-yl | cyclopropyl |
| 28. | N, N-diethylamino | cyclopropyl |
| 29. | N-isopropylamino | cyclopropyl |
| 30. | N-ethylamino | cyclopropyl |
| 31. | N,N-methyl-isopropylamino | cyclopropyl |
| 32. | acetidin-1-yl | cyclopropyl |
| 33. | morpholin-4-yl | cyclopropyl |
| 34. | N-cyclopentylamino | cyclopropyl |
| 35. | [1-(4-fluorophenyl)ethyl]amino | cyclopropyl |
| 36. | (cyclopropylmethyl)amino | cyclopropyl |
| 37. | methyl | cyclopropyl |
| 38. | 8-methyl-3,8- | cyclopropyl |
| | diazabicyclo[3.2.1]octan-3-yl | |
| 39. | 1-methylpiperidin-4-yl | cyclopropyl |
| 40. | thiomorpholine-4-yl-1,1dioxide | cyclopropyl |
| 41. | 3-(dimethylamino)azetidin-1-yl | cyclopropyl |
| 42. | 4-(dimethylamino)piperidin-1-yl | cyclopropyl |
| 43. | N-ethan-1-ol-amino | cyclopropyl |
| 44. | azetidine-3-carbonyloxy methyl | cyclopropyl |
| 45. | N,N-dimethylamino methyl | cyclopropyl |
| 46. | 2-oxa-6-azaspiro[3.3]heptan-6-yl | cyclopropyl |
| 47. | pyrrolidine-3-yl | cyclopropyl |
| 48. | (piperazin-1-yl)ethyl | cyclopropyl |
| 49. | N,N-dimethylamino | CN |
| 50. | 4-methyl-piperazin-1-yl | CN |
| 51. | pyrrolidin-1-yl | CN |
| 52. | N,N-diethylamino | CN |
| 53. | N-isopropylamino | CN |
| 54. | N-ethylamino | CN |
| 55. | N,N-methyl-isopropylamino | CN |
| 56. | acetidin-1-yl | CN |
| 57. | morpholin-4-yl | CN |
| 58. | N-cyclopentylamino | CN |
| 59. | [1-(4-fluorophenyl)ethyl]amino | CN |
| 60. | (cyclopropylmethyl)amino | CN |
| 61. | methyl | CN |
| 62. | 8-methyl-3,8- | CN |
| | diazabicyclo[3.2.1]octan-3-yl | |
| 63. | 1-methylpiperidin-4-yl | CN |
| 64. | thiomorpholine-4-yl-1,1dioxide | CN |
| 65. | 3-(dimethylamino)azetidin-1-yl | CN |
| 66. | 4-(dimethylamino)piperidin-1-yl | CN |
| 67. | N-ethan-1-ol-amino | CN |
| 68. | azetidine-3-carbonyloxy methyl | CN |
| 69. | N,N-dimethylamino methyl | CN |
| 70. | 2-oxa-6-azaspiro[3.3]heptan-6-yl | CN |
| 71. | pyrrolidine-3-yl | CN |
| 72. | (piperazin-1-yl)ethyl | CN |

Another special embodiment are the compounds of formula (I-A.a′)

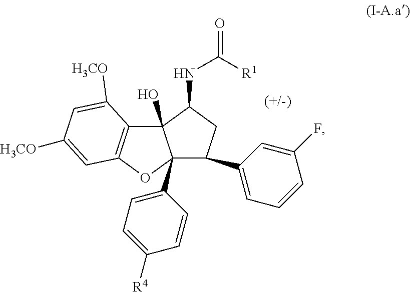

wherein R¹ and R⁴ have one of the meanings selected from the definition given in one line of table 1 above.

Another special embodiment are the compounds of formula (I-A.a)

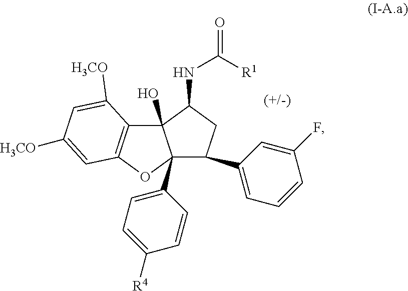

wherein R¹ and R⁴ have one of the meanings selected from the definition given in one line of table 1 above.

Another special embodiment are the compounds of formula of formula (I), an enantiomeric mixture comprising the compounds of formula (I-A.a) and (I-A.b) or the pharmaceutically acceptable salt thereof,

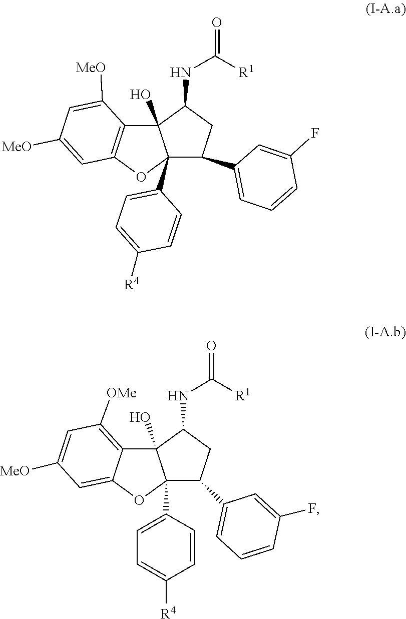

wherein R¹ and R⁴ have one of the meanings selected from the definition given in one line of table 1 above.

In particular compound of formula (I) is a mixture of (I-A.a) and (I-A.b), wherein the enantiomer excess (ee) of the enantiomer of formula (I-A.a) is at least 20%, preferably at least 50%, in particular at least 80%, especially at least 99% and wherein R¹ and R⁴ have one of the meanings selected from the definition given in one line of table 1 above.

Another special embodiment are the compounds selected from A, B, C, D, E, F, G, H, I, J, K, L, M, N, O, P, Q, R, S, T, U, V, W and the mixture of the each of compounds A to V with its respective enantiomer:

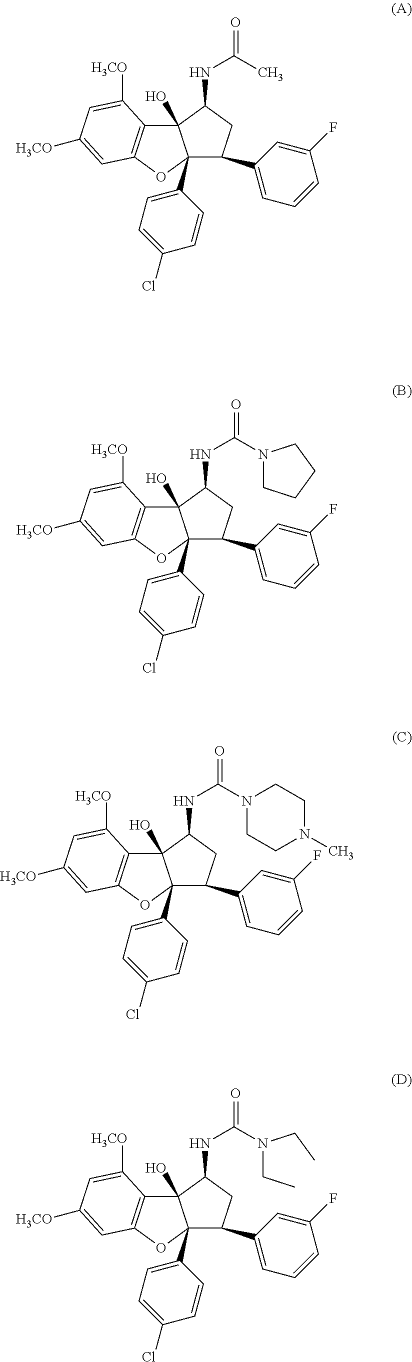 [SMILES: COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@@]1(c3ccc(Cl)cc3)[C@H](c3cccc(F)c3)C[C@H](NC(=O)N3CCCC3)[C@@]21O ; COc1cc(OC)c2c(c1)O[C@@]1(c3ccc(Cl)cc3)[C@H](c3cccc(F)c3)C[C@H](NC(=O)N3CCN(C)CC3)[C@@]21O ; CCN(CC)C(=O)N[C@H]1C[C@@H](c2cccc(F)c2)[C@]2(c3ccc(Cl)cc3)Oc3cc(OC)cc(OC)c3[C@]12O]

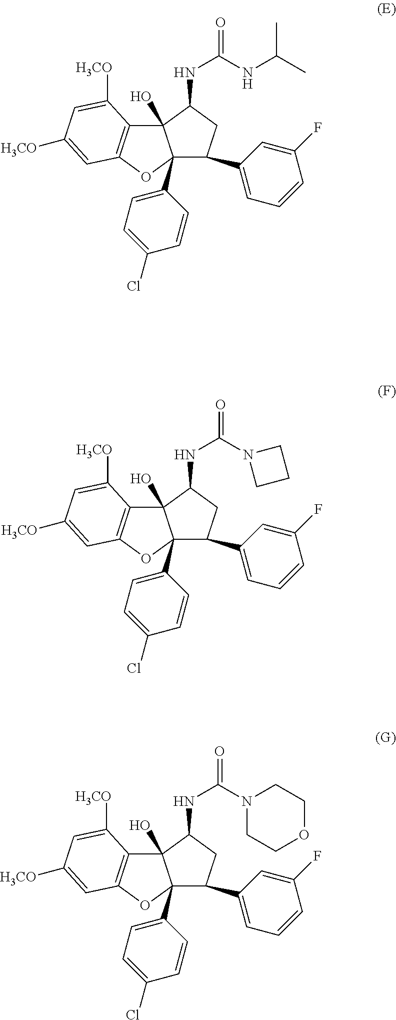 [SMILES: COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@@]1(c3ccc(Cl)cc3)[C@H](c3cccc(F)c3)C[C@H](NC(=O)N3CCC3)[C@@]21O ; COc1cc(OC)c2c(c1)O[C@@]1(c3ccc(Cl)cc3)[C@H](c3cccc(F)c3)C[C@H](NC(=O)N3CCOCC3)[C@@]21O]

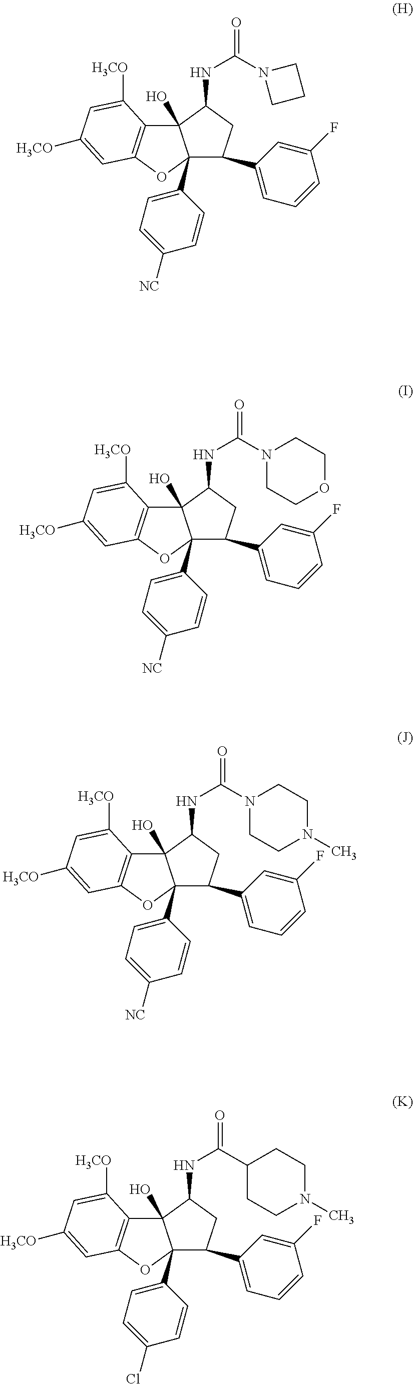 [SMILES: COc1cc(OC)c2c(c1)O[C@@]1(c3ccc(C#N)cc3)[C@H](c3cccc(F)c3)C[C@H](NC(=O)N3CCC3)[C@@]21O ; COc1cc(OC)c2c(c1)O[C@@]1(c3ccc(C#N)cc3)[C@H](c3cccc(F)c3)C[C@H](NC(=O)N3CCOCC3)[C@@]21O ; COc1cc(OC)c2c(c1)O[C@@]1(c3ccc(C#N)cc3)[C@H](c3cccc(F)c3)C[C@H](NC(=O)N3CCN(C)CC3)[C@@]21O ; COc1cc(OC)c2c(c1)O[C@@]1(c3ccc(Cl)cc3)[C@H](c3cccc(F)c3)C[C@H](NC(=O)C3CCN(C)CC3)[C@@]21O]

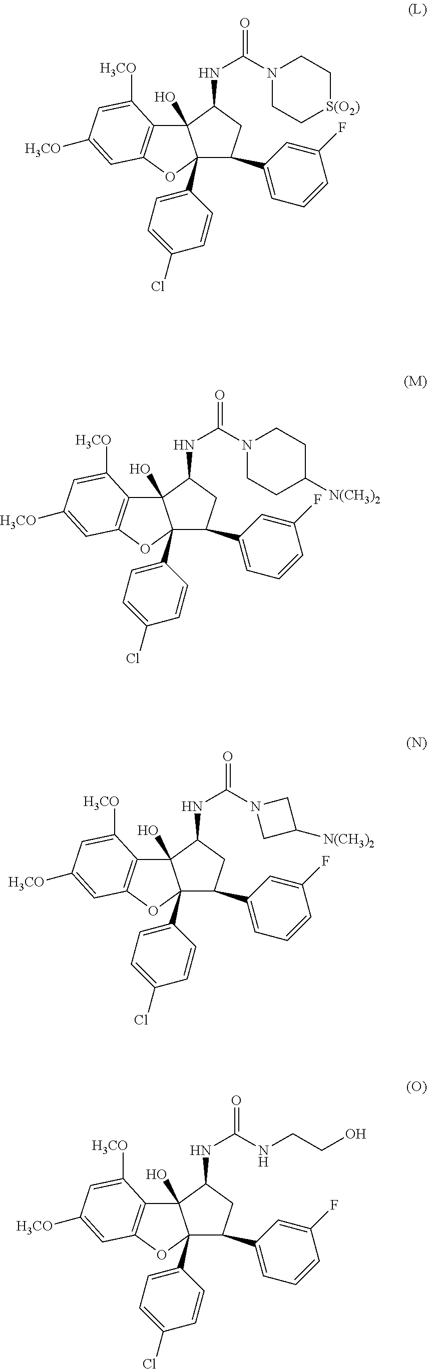

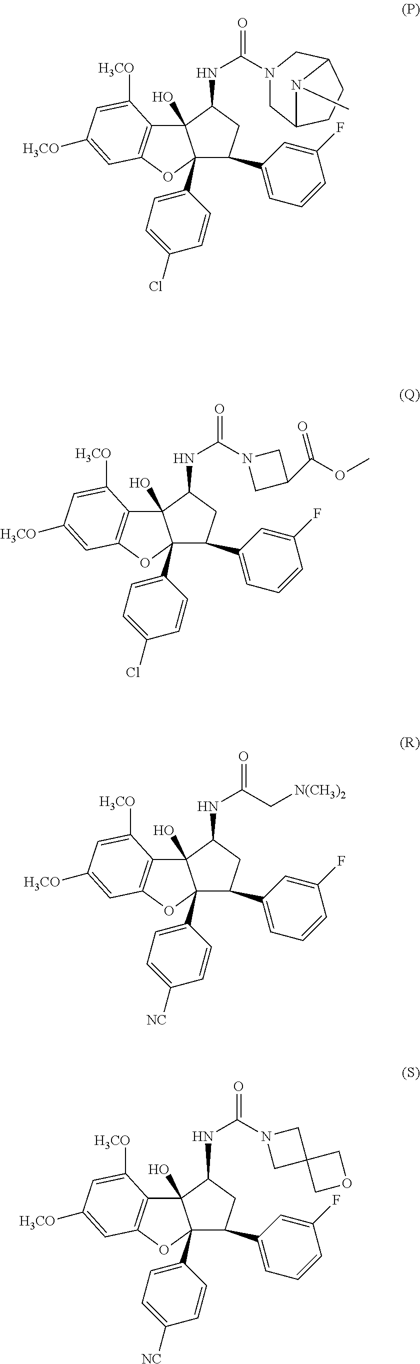 [SMILES: COc1cc(OC)c2c(c1)O[C@@]1(c3ccc(Cl)cc3)[C@H](c3cccc(F)c3)C[C@H](NC(=O)N3CC4CCC(C3)N4C)[C@@]21O ; COC(=O)C1CN(C(=O)N[C@H]2C[C@@H](c3cccc(F)c3)[C@]3(c4ccc(Cl)cc4)Oc4cc(OC)cc(OC)c4[C@]23O)C1 ; COc1cc(OC)c2c(c1)O[C@@]1(c3ccc(C#N)cc3)[C@H](c3cccc(F)c3)C[C@H](NC(=O)CN(C)C)[C@@]21O ; COc1cc(OC)c2c(c1)O[C@@]1(c3ccc(C#N)cc3)[C@H](c3cccc(F)c3)C[C@H](NC(=O)N3CC4(COC4)C3)[C@@]21O]

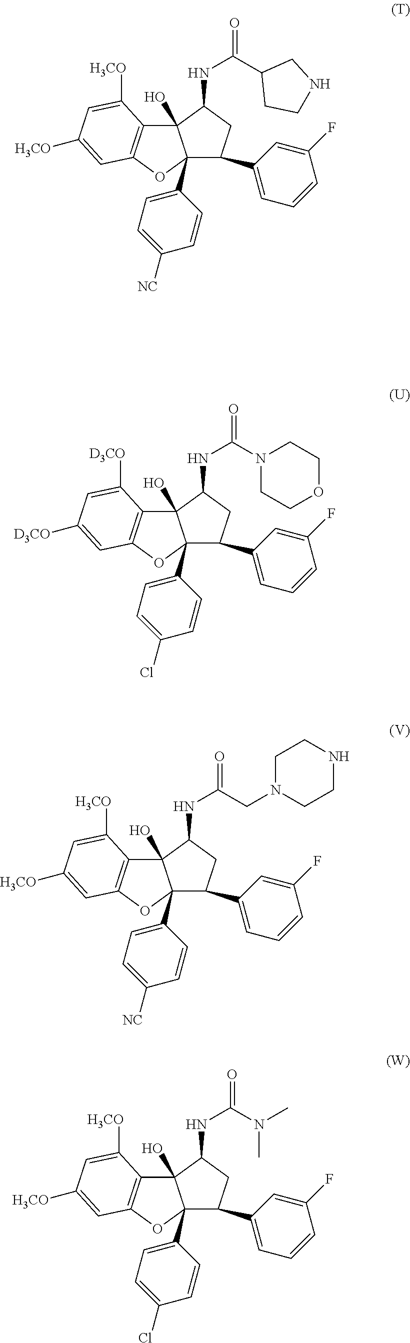

or mixtures thereof.

Preferred is a compound of formula (A) or an enantiomeric mixture comprising the compounds of formula (A) and its enantiomer, in particular wherein the enantiomer excess (ee) of the enantiomer of formula (A) is at least 20%, preferably at least 50%, in particular at least 80%, especially at least 99%.

Preferred is a compound of formula (B) or an enantiomeric mixture comprising the compounds of formula (B) and its enantiomer, in particular wherein the enantiomer excess (ee) of the enantiomer of formula (B) is at least 20%, preferably at least 50%, in particular at least 80%, especially at least 99%.

Preferred is a compound of formula (C) or an enantiomeric mixture comprising the compounds of formula (C) and its enantiomer, in particular wherein the enantiomer excess (ee) of the enantiomer of formula (C) is at least 20%, preferably at least 50%, in particular at least 80%, especially at least 99%.

Preferred is a compound of formula (D) or an enantiomeric mixture comprising the compounds of formula (D) and its enantiomer, in particular wherein the enantiomer excess (ee) of the enantiomer of formula (D) is at least 20%, preferably at least 50%, in particular at least 80%, especially at least 99%.

Preferred is a compound of formula (E) or an enantiomeric mixture comprising the compounds of formula (E) and its enantiomer, in particular wherein the enantiomer excess (ee) of the enantiomer of formula (E) is at least 20%, preferably at least 50%, in particular at least 80%, especially at least 99%.

Preferred is a compound of formula (F) or an enantiomeric mixture comprising the compounds of formula (F) and its enantiomer, in particular wherein the enantiomer excess (ee) of the enantiomer of formula (F) is at least 20%, preferably at least 50%, in particular at least 80%, especially at least 99%.

Preferred is a compound of formula (G) or an enantiomeric mixture comprising the compounds of formula (G) and its enantiomer, in particular wherein the enantiomer excess (ee) of the enantiomer of formula (G) is at least 20%, preferably at least 50%, in particular at least 80%, especially at least 99%.

Preferred is a compound of formula (H) or an enantiomeric mixture comprising the compounds of formula (H) and its enantiomer, in particular wherein the enantiomer excess (ee) of the enantiomer of formula (H) is at least 20%, preferably at least 50%, in particular at least 80%, especially at least 99%.

Preferred is a compound of formula (I) or an enantiomeric mixture comprising the compounds of formula (I) and its enantiomer, in particular wherein the enantiomer excess (ee) of the enantiomer of formula (I) is at least 20%, preferably at least 50%, in particular at least 80%, especially at least 99%.

Preferred is a compound of formula (J) or an enantiomeric mixture comprising the compounds of formula (J) and its enantiomer, in particular wherein the enantiomer excess (ee) of the enantiomer of formula (J) is at least 20%, preferably at least 50%, in particular at least 80%, especially at least 99%.

Preferred is a compound of formula (K) or an enantiomeric mixture comprising the compounds of formula (K) and its enantiomer, in particular wherein the enantiomer excess (ee) of the enantiomer of formula (K) is at least 20%, preferably at least 50%, in particular at least 80%, especially at least 99%.

Preferred is a compound of formula (L) or an enantiomeric mixture comprising the compounds of formula (L) and its enantiomer, in particular wherein the enantiomer excess (ee) of the enantiomer of formula (L) is at least 20%, preferably at least 50%, in particular at least 80%, especially at least 99%.

Preferred is a compound of formula (M) or an enantiomeric mixture comprising the compounds of formula (M) and its enantiomer, in particular wherein the enantiomer excess (ee) of the enantiomer of formula (M) is at least 20%, preferably at least 50%, in particular at least 80%, especially at least 99%.

Preferred is a compound of formula (N) or an enantiomeric mixture comprising the compounds of formula (N) and its enantiomer, in particular wherein the enantiomer excess (ee) of the enantiomer of formula (N) is at least 20%, preferably at least 50%, in particular at least 80%, especially at least 99%.

Preferred is a compound of formula (O) or an enantiomeric mixture comprising the compounds of formula (O) and its enantiomer, in particular wherein the enantiomer excess (ee) of the enantiomer of formula (O) is at least 20%, preferably at least 50%, in particular at least 80%, especially at least 99%.

Preferred is a compound of formula (Q) or an enantiomeric mixture comprising the compounds of formula (Q) and its enantiomer, in particular wherein the enantiomer excess (ee) of the enantiomer of formula (Q) is at least 20%, preferably at least 50%, in particular at least 80%, especially at least 99%.

Preferred is a compound of formula (R) or an enantiomeric mixture comprising the compounds of formula (R) and its enantiomer, in particular wherein the enantiomer excess (ee) of the enantiomer of formula (R) is at least 20%, preferably at least 50%, in particular at least 80%, especially at least 99%.

Preferred is a compound of formula(S) or an enantiomeric mixture comprising the compounds of formula(S) and its enantiomer, in particular wherein the enantiomer excess (ee) of the enantiomer of formula(S) is at least 20%, preferably at least 50%, in particular at least 80%, especially at least 99%.

Preferred is a compound of formula (T) or an enantiomeric mixture comprising the compounds of formula (T) and its enantiomer, in particular wherein the enantiomer excess (ee) of the enantiomer of formula (T) is at least 20%, preferably at least 50%, in particular at least 80%, especially at least 99%.

Preferred is a compound of formula (U) or an enantiomeric mixture comprising the compounds of formula (U) and its enantiomer, in particular wherein the enantiomer excess (ee) of the enantiomer of formula (U) is at least 20%, preferably at least 50%, in particular at least 80%, especially at least 99%.

Preferred at least a compound of formula (V) or an enantiomeric mixture comprising the compounds of formula (V) and its enantiomer, in particular wherein the enantiomer excess (ee) of the enantiomer of formula (V) is at least 20%, preferably at least 50%, in particular at least 80%, especially at least 99%.

Preferred at least a compound of formula (W) or an enantiomeric mixture comprising the compounds of formula (W) and its enantiomer, in particular wherein the enantiomer excess (ee) of the enantiomer of formula (W) is at least 20%, preferably at least 50%, in particular at least 80%, especially at least 99%.

In one embodiment the compounds, wherein
R¹ is NH₂, R⁴ is Cl, R⁶ is F, R⁷ is hydrogen, R⁸ is OCH₃, R⁹ is OCH₃,
R¹ is N(CH₃)₂, R⁴ is CI, R^{e} is F, R⁷ is hydrogen, R⁸ is OCH₃, R⁹ is OCH₃,
are excluded from the invention.

### Virus

The present invention provides a compound of formula (I) as defined herein and pharmaceutically acceptable salts thereof (“compounds of the invention”) for use in treatment or prevention of diseases caused by virus infections or diseases associated with virus infections.

In the sense of the invention the term “diseases caused by virus infections” also denoted as “primary diseases” relates to diseases, which immediately result from a virus infection or are the manifestation of a disease in a series of successive diseases (in the following denoted as associated diseases).

In the sense of the invention the term “diseases associated with virus infections” also denoted as “secondary diseases” firstly relates to diseases in which a further pathogen, such as bacteria, allergen, fungicide, virus different from the virus of the first virus infection, attacks an organism in which a primary disease has already occurred (“first virus infection”). Diseases associated with virus infections (secondary disease) often occur when the immune system is weakened by the primary diseases or the entry barriers for pathogens are damaged by the primary disease. In some diseases, the treatment of secondary diseases can be the most urgent therapeutic problem, e.g., in the case of AIDS. Secondly, the term “secondary diseases” refers to follow-up diseases that result from a primary disease. This encompasses e.g., cardiologic, neurological complications as defined in the following.

Preferably, the present invention provides a compound of formula (I) as defined herein and pharmaceutically acceptable salts thereof for use in treatment or prevention of diseases caused by virus infections or diseases associated with virus infections, wherein the disease is selected from virus infections caused by positive single-stranded RNA viruses (+ssRNA viruses) and a negative single-stranded RNA viruses (-ssRNA viruses), in particular for use in treatment or prevention of diseases, wherein the disease is selected from virus infection caused by positive single-stranded RNA viruses ((+) ssRNA) and negative single-stranded RNA viruses ((−) ssRNA).

Positive-strand RNA viruses (+ssRNA viruses) are a group of related viruses that have positive-sense, single-stranded genomes made of ribonucleic acid. The positive-sense genome can act as messenger RNA (mRNA) and can be directly translated into viral proteins by the host cell's ribosomes. Positive-strand RNA viruses encode an RNA-dependent RNA polymerase (RdRp) which is used during replication of the genome to synthesize a negative-sense antigenome that is then used as a template to create a new positive-sense viral genome.

Especially, the (+) ssRNA virus is a member of a family selected from togaviridae, flaviviridae, coronaviridae and retroviridae.

Togaviridae is a family of enveloped positive-strand RNA viruses. It currently includes two genuses of enveloped viruses with a single-stranded RNA with positive polarity. The togaviridae include virus species in the genus alphavirus and rubella virus. Alphavirus transmits diseases to humans and animals through insects. Rubella virus transmite only between humans via the respiratory route.

Flaviviridae is a family of enveloped positive-strand RNA viruses which mainly infect mammals and birds. They are primarily spread through arthropod vectors (mainly ticks and mosquitoes). The flaviviridae include virus species in the genus hepaciviruses and flaviviruses. Diseases associated with this group include hepatitis, Dengue fever, Japanese encephalitis, Kyasanur Forest disease, Powassan virus, West Nile fever, Yellow fever and Zika fever.

Coronavirus is the common name for Coronaviridae and Orthocoronavirinae, also called Coronavirinae. Coronaviridae is a family of enveloped, positive-strand RNA viruses. Coronaviruses cause diseases in mammals and birds. In humans, the viruses cause respiratory infections.

The coronaviridae include virus species in the genus alphacoronavirus, betacoronavirus and torovirus. Preferably, the coronaviridae is selected from SARS-COV, SARS-COV-2, MERS-COV, HCoV-229E, HCoV-NL63, HCoVC43 and HKU1.

Four human coronaviruses cause typically minor symptoms of a common cold, while three are known to cause more serious illness and can be lethal: SARS-COV-1, which causes SARS; MERS-COV, which causes MERS; and SARS-COV-2, which causes COVID-19.

Severe acute respiratory syndrome coronavirus 1 (SARS-COV-1 or SARS-COV) is a strain of coronavirus that causes severe acute respiratory syndrome (SARS). It causes an often severe illness and is marked initially by systemic symptoms of muscle pain, headache, and fever, followed in 2-14 days by the onset of respiratory symptoms, mainly cough, dyspnea, and pneumonia. Another common finding in SARS patients is a decrease in the number of lymphocytes circulating in the blood. SARS-COV is a member of the genus betacoronavirus and subgenus embecovirus.

Severe acute respiratory syndrome coronavirus 2 (SARS-COV-2) is a strain of coronavirus that causes COVID-19, a respiratory illness. SARS-COV-2 is a virus of the species severe acute respiratory syndrome-related coronavirus (SARSr-COV), related to the SARS-COV-1 virus. SARS-COV-2 is a member of the genus betacoronavirus and subgenus embecovirus.

Middle East respiratory syndrome-related coronavirus (MERS-COV) is the virus that causes Middle East respiratory syndrome (MERS). It is a species of coronavirus which infects humans, bats, and camels. The species is a member of the genus Betacoronavirus and subgenus Merbecovirus.

Human coronavirus 229E (HCoV-229E) is a species of coronavirus which infects humans and bats. It is one of the viruses responsible for the common cold. HCoV-229E is a member of the genus alphacoronavirus and subgenus duvinacovirus.

Human coronavirus NL63 (HCoV-NL63) is a species of coronavirus, specifically a Setracovirus from among the Alphacoronavirus genus. Infection with the virus has been confirmed worldwide, and has an association with many common symptoms and diseases. Associated diseases include mild to moderate upper respiratory tract infections, severe lower respiratory tract infections, croup and bronchiolitis.

Human coronavirus OC43 (HCoV-OC43) is a member of the species Betacoronavirus 1, which infects humans and cattle. OC43 is one of seven coronaviruses known to infect humans. It is one of the viruses responsible for the common cold. It is of the genus betacoronavirus and subgenus embecovirus.

Human coronavirus HKU1 (HCoV-HKU1) is a species of coronavirus in humans and animals. It causes an upper respiratory disease with symptoms of the common cold, but can advance to pneumonia and bronchiolitis. HCoV-HKU1 is a member of the genus betacoronavirus and subgenus embecovirus.

Retroviridae is a family of enveloped positive-strand RNA viruses. The retroviridae include virus species in the genus inter alia deltretrovirus and lentivirus. Lentivirus causes chronic and deadly diseases characterized by long incubation periods, in humans and other mammalian species. The genus includes the human immunodeficiency virus (HIV), which causes AIDS.

Negative-strand RNA viruses (-ssRNA viruses) are a group of related viruses that have negative-sense, single-stranded genomes made of ribonucleic acid. They have genomes that act as complementary strands from which messenger RNA (mRNA) is synthesized by the viral enzyme RNA-dependent RNA polymerase (RdRp). During replication of the viral genome, RdRp synthesizes a positive-sense antigenome that it uses as a template to create genomic negative-sense RNA. Negative-strand RNA viruses also share a number of other characteristics: most contain a viral envelope that surrounds the capsid, which encases the viral genome, −ssRNA virus genomes are usually linear, and it is common for their genome to be segmented.

Preferably, the (−) ssRNA virus is a member of a family selected from arenaviridae, bornaviridae, bunyviridae, filoviridae, othymyxoviridae, paramyxoviridae and pneumoviridaerhabdoviridae, in particular filoviridae.

Preferably, filoviridae is selected from Bundibugyo ebolavirus, Reston ebolavirus, Sudan ebolavirus, Taï Forest ebolavirus, Zaire ebolavirus and Bombali ebolavirus.

As defined above positive single-stranded RNA viruses ((+) ssRNA) and negative single-stranded RNA viruses ((−) ssRNA) account for a large fraction of known viruses, including many pathogens as well as less clinically serious pathogens such as the rhinoviruses that cause the common cold. Therefore, in one embodiment the disease is a disease associated with positive single-stranded RNA viruses ((+) ssRNA) and negative single-stranded RNA viruses ((−) ssRNA).

In a preferred embodiment, the compounds of formula (I) are for use in treatment or prevention of diseases caused by virus infections, in in particular for use in treatment or prevention of diseases, wherein the disease is selected from virus infection caused by positive single-stranded RNA viruses ((+) ssRNA) and negative single-stranded RNA viruses ((−) ssRNA), especially, wherein the disease is selected from dengue fever, MERS, COVID-19, SARS, ebobla fever, AIDS and zika fever.

In a preferred embodiment, the compounds of formula (I) are for use in treatment or prevention of diseases associated with virus infections.

Diseases associated with positive-sense single-stranded RNA virus infections and negative-sense single-stranded RNA virus infections include various complications arising therefrom. Complications include respiratory distress, pulmonary fibrosis, pneumonia, cytokine storm, acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events, rhabdomyolysis, neurocognitive deficits, and sensory and motor deficits. The present invention embraces the complications caused by SARS-COV-2 infection.

In one embodiment the disease is lung inflammation. Suitably, the lung inflammation is caused by pathogenic infection, bacterial infection, fungal infection or viral infection, in particular a (+) ssRNA virus infection. More suitably, the lung inflammation is caused by a disease selected from the group consisting of pneumonia, acute respiratory disease symptom (ARDS), CORD, asthma, idiopathic pulmonary fibrosis, allergic rhinitis, rhinitis and sinusitis. More suitably, the lung inflammation is caused by CORD, asthma or idiopathic pulmonary fibrosis. Even more suitably, the lung inflammation is caused by CORD. Even more suitably, the lung inflammation is caused by asthma. Even more suitably, the lung inflammation is caused by idiopathic pulmonary fibrosis.

In one embodiment, the compounds of the invention are used for the treatment of hyperinflammation associated with positive-sense single-stranded RNA virus infections, such as coronavirus infection e.g. the compounds of the invention reduce hyperinflammation associated with coronavirus infection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “therapeutically effective” is intended to qualify the amount of each agent, which will achieve the goal of improvement in disorder severity and the frequency of incidence, while avoiding adverse side-effects typically associated with alternative therapies. For example, effective anticancer agents prolong the survivability of the patient or his/her life quality, inhibit the rapidly proliferating cell growth associated with the neoplasm, or effect a regression of the neoplasm.

The terms “treat,” “treating,” and “treatment,” as used herein, refer to any type of intervention or process performed on, or administering an active agent to, the subject with the objective of reversing, alleviating, ameliorating, inhibiting or slowing down or preventing the progression, development, severity or recurrence of a symptom, complication, condition or biochemical indicia associated with a disease. By contrast, “prophylaxis” or “prevention” refers to administration to a subject who does not have a disease to prevent the disease from occurring.

As used herein, the term “cell” is meant to refer to a cell that is in vitro, ex vivo or in vivo. In the sense of the invention, an ex vivo cell can be part of a tissue sample excised from an organism such as a mammal. In the sense of the invention, an in vitro cell can be a cell in a cell culture. In the sense of the invention, an in vivo cell is a cell living in an organism such as a mammal.

The term “patient” includes humans and animals that receive either therapeutic or prophylactic treatment.

The term “subject” includes any human or animal. For example, the methods and compositions herein disclosed can be used to treat a subject having cancer.

A (non-human) animal includes all vertebrates, e.g. mammals and non-mammals, including cows, sheep, pigs, goats, horses, poultry, dogs, cats, non-human primates, rodents etc. In one embodiment, the subject is a human subject.

The phrase “pharmaceutically acceptable carrier” as used herein means a pharmaceutically acceptable material, composition or vehicle, such as a liquid or solid diluent, solvent, excipient, manufacturing aid (e.g. lubricant) or encapsulating material, involved in carrying or transporting the subject compound from one organ, or portion of the body, to another organ, or portion of the body. Each carrier must be “acceptable” in the sense of being compatible with the other ingredients of the formulation.

Suitable other ingredients are the afore-mentioned carrier and further additives, including adjuvants, preserving agents, fillers, flow regulating agents, disintegrating agents, wetting agents, emulsifying agents, suspending agents, sweetening agents, flavoring agents, bittering agents, perfuming agents, antibacterial agents, antifungal agents, lubricating agents, dispensing agents, etc., Suitable additives are selected depending on the nature of the mode of administration and dosage forms; and not injurious to the patient.

The term “pharmaceutical composition” means a composition comprising a compound of the invention in combination with at least one further compound selected from
a) at least one further pharmaceutically active substance and
b) at least one additional pharmaceutically acceptable carrier and or additive.

For use in therapy the compounds of formula (I) are usually administered as a pharmaceutical composition. The invention also relates to a pharmaceutical composition comprising a compound of formula (I) or a pharmaceutically acceptable salt thereof and a pharmaceutically acceptable carrier.

The compounds of the invention may be administered by any convenient method, e.g. by oral, parenteral, buccal, sublingual, nasal, rectal, intrathecal or transdermal administration or by inhalation (e.g. for topical administration to the lung by inhalation), and the pharmaceutical compositions adapted accordingly.

A compound of of formula (I) which is active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be optimized and then reconstituted with a suitable solvent just prior to administration.

Compositions for nasal administration or for inhalation (e.g. for topical administration to the lung by inhalation) may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve suitably for delivery of the aerosol to the nasal or bronchial passages. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Topical administration to the lung may also be achieved by use of a dry-powder formulation which contains the compound of the invention in finely divided form optionally together with one or more carriers or other excipients. A dry powder formulation is typically delivered using a dry powder inhaler (DPI) device.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. In one embodiment the composition is in unit dose form such as a tablet, capsule or ampoule.

In one embodiment of the invention, the compound of formula (I) is used in combination with a further therapeutic agent or agents. When the compound of formula (I) are used in combination with other therapeutic agents, the compound of formula (I) may be administered either sequentially or simultaneously by any convenient route. Alternatively, the compounds may be administered separately.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

FIG. 1: The mRNA of Dual reporter assay is shown. Firefly luciferase (F Luc) will be translated by elF4F complex and Renilla Luciferase (R Luc) will be translated independently of the complex.

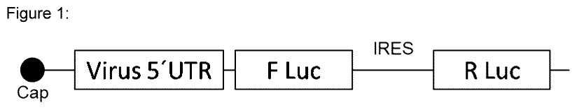

FIG. 2: The sequence of 5′UTR of mRNA of Virus are shown.

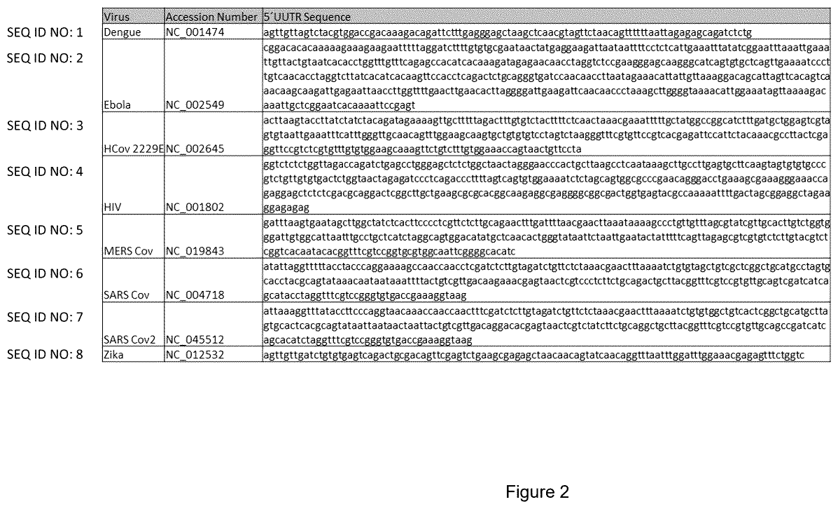

FIG. 3: the dual luciferase assay for Cap-dependent translation initiation is shown. A Plasmid MAP of the dual luciferase assay system. B Dual luciferase assay for Cap-dependent translation initiation was performed in Hela cells transfected with dual luciferase reporter gene based on pFR_HCV_xb. Cells were treated with compounds 1, 2, 3, 4 (100 nM) for 24 h in serum-free DMEM. After incubation, dual luciferase reporter assay was performed according to the manufacturer's instruction and the luminescence was measured using a multiplate reader. Data were normalized to cells transfected with the indicated mutant and exposed to DMSO for 24 h. DMSO-treated cells were set as 1. The bars represent mean±SEM from 3 independent experiments. The dot plot indicate the value from each experiment. (EV corresponds to empty vector)

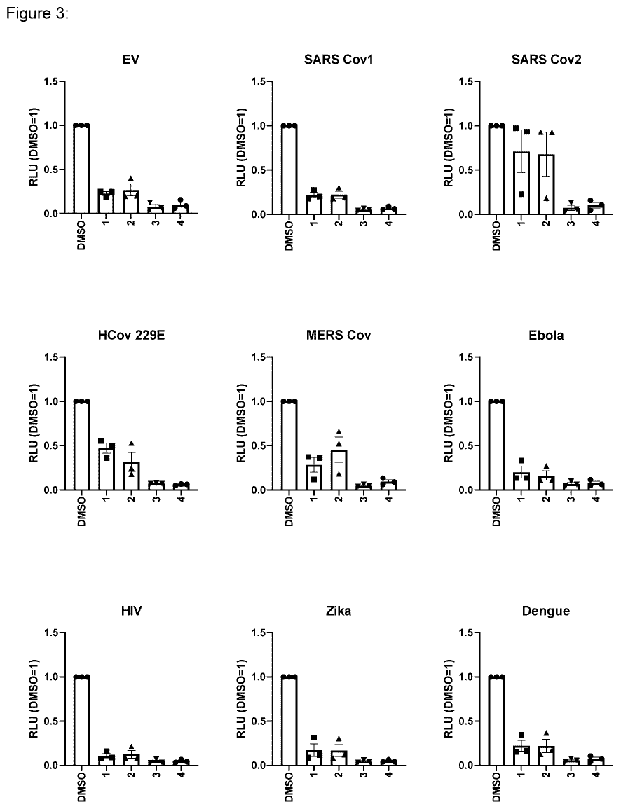

### EXAMPLES

The compound 1, 2, 3 and 4 can be prepared analogously to PCT/EP 2022/025396

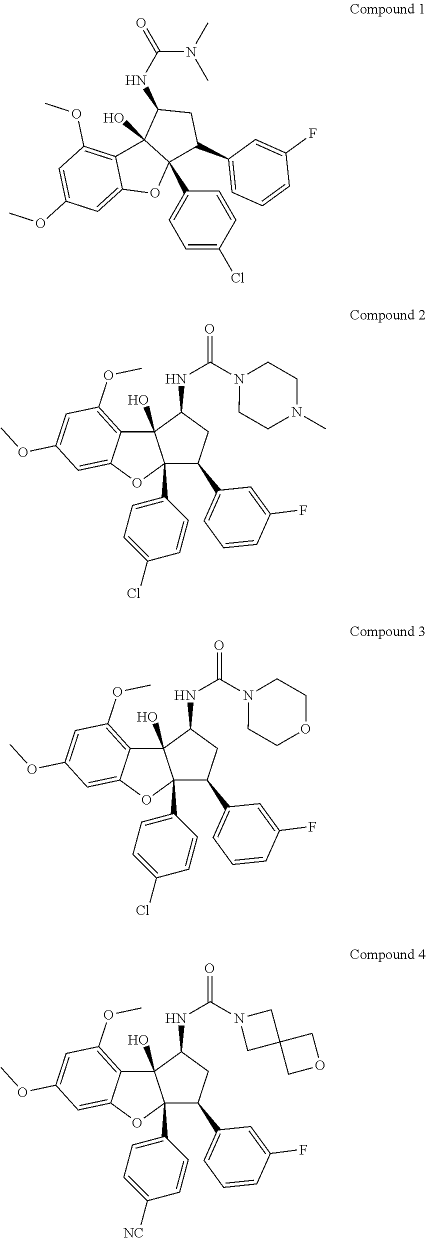

1. A process for use in treatment or prevention of a disease caused by a virus infection in a subject in need thereof, wherein the disease results from a virus infection caused by a positive single-stranded RNA virus ((+) ssRNA) or a negative single-stranded RNA virus ((−) ssRNA), comprising administering to the subject a compound of formula (I) 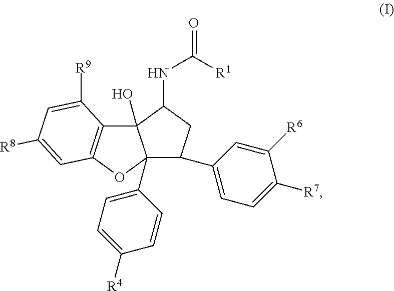
or a pharmaceutically acceptable salt thereof, wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R′;
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R⁴ is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R^{e} is F, R⁷ is hydrogen;
R& is selected from OCH₃, OCD₃; and
R⁹ is selected from OCH₃, OCD₃.

2. The process according to claim 1, wherein compound (I) comprises a mixture of (I.a) and (I.b) or a mixture of the pharmaceutically acceptable salts thereof, wherein an enantiomer excess (ee) of the enantiomer of formula (I.a) is at least 20%

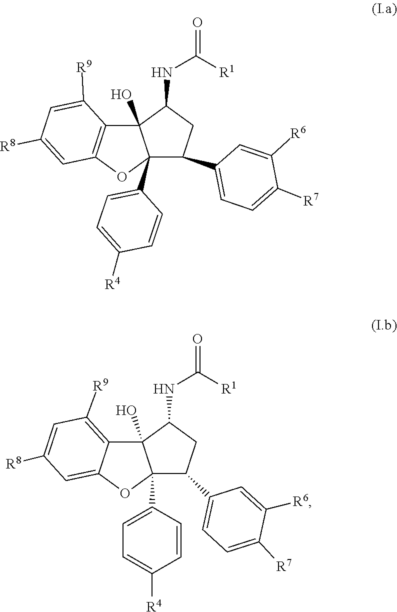

3. The process of claim 1, wherein R⁶ is F and R⁷ is hydrogen.

4. The process of claim 1, wherein R¹ is selected from
C₁-C₄ alkyl, which is unsubstituted or substituted by 1, 2 or 3 substituents R^{a}; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c}, S, SO and SO₂, wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi-or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}; or
R² and R³ together with the nitrogen atom, which they are attached to, form a saturated or partly unsaturated spiro moiety, comprising 1 or 2 identical or different heteroatoms or heteroatom-containing groups as ring members, selected from N, NR^{c} and O and wherein the heterocyclic ring is unsubstituted or substituted by 1, 2 or 3 identical or different radicals R^{g}.

5. The process of claim 1, wherein R¹ is selected from
NR²R³, wherein R² and R³ independently from each other are selected from hydrogen, C₁-C₃ alkyl and C₃-C₆ cycloalkyl, wherein alkyl is unsubstituted or substituted by 1 or 2 substituents R^{a}, wherein cycloalkyl is unsubstituted or substituted by 1 or 2 substituents R^{b}, preferably R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{e} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl)₂, NH(C₁-C₂ alkyl) and carbonyloxy-C₁-C₄-alkyl, preferably R² and R³ together with the nitrogen atom, which they are attached to, form a pyrrolidine ring, piperazine ring, azetidine ring or morpholine ring, wherein the pyrrolidine ring, piperazine ring, azetidine ring, or morpholine ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) and carbonyloxy-C₁-C₄-alkyl; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR_{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably R² and R³ together with the nitrogen atom, which they are attached to, form a 2-oxa-6-azaspiro[3.3]heptan-6-yl spiro compound.

6. The process of claim 1, wherein R¹ is a C₅-C₇ heterocyloalkyl comprising 1 or 2 heteroatoms or heteroatom-containing groups as ring members, NR^{c} or S, wherein the heterocyloalkyl is connected to via a carbon atom.

7. The process of claim 1, wherein R¹ is a methyl, 4-methyl-piperazin-1-yl, pyrrolidin-1-yl, N,N-diethylamino, N-isopropylamino, N-ethylamino, N,N-methyl-isopropylamino, azetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1,1-dioxide, 3-(dimethylamino) azetidin-1-yl, 4-(dimethylamino) piperidin-1-yl, N-ethan-1-ol-amino, azetidine-3-carbonyloxymetyl, N,N-dimethylamino-methyl, 2-oxa-6-azaspiro[3.3]heptan-6-yl or pyrrolidine-3-yl.

8. The process of claim 1, wherein the compound of formula (1) is formulae A, B, C, D, E, F, G, H, I, J, K, L, M, N, O, P, Q, R, S, T, U, V, W, or a mixture with a respective enantiomer 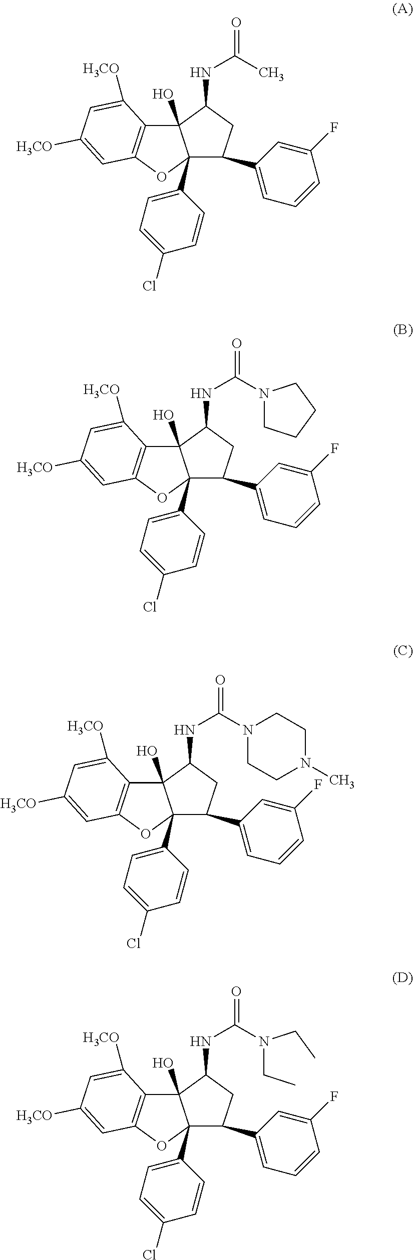 [SMILES: COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@@]1(c3ccc(Cl)cc3)[C@H](c3cccc(F)c3)C[C@H](NC(=O)N3CCCC3)[C@@]21O ; COc1cc(OC)c2c(c1)O[C@@]1(c3ccc(Cl)cc3)[C@H](c3cccc(F)c3)C[C@H](NC(=O)N3CCN(C)CC3)[C@@]21O ; CCN(CC)C(=O)N[C@H]1C[C@@H](c2cccc(F)c2)[C@]2(c3ccc(Cl)cc3)Oc3cc(OC)cc(OC)c3[C@]12O] 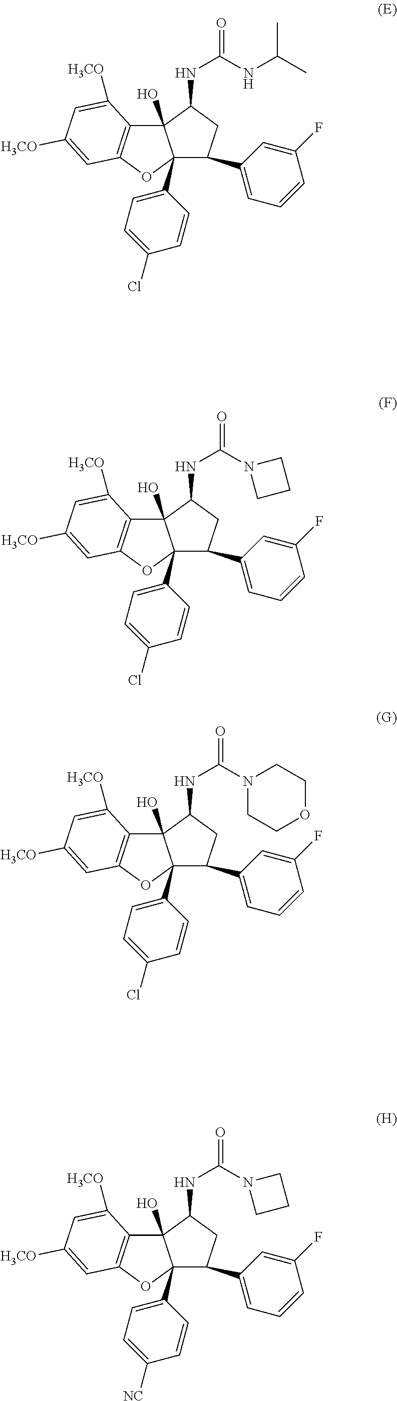 [SMILES: COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@@]1(c3ccc(Cl)cc3)[C@H](c3cccc(F)c3)C[C@H](NC(=O)N3CCC3)[C@@]21O ; COc1cc(OC)c2c(c1)O[C@@]1(c3ccc(Cl)cc3)[C@H](c3cccc(F)c3)C[C@H](NC(=O)N3CCOCC3)[C@@]21O ; COc1cc(OC)c2c(c1)O[C@@]1(c3ccc(C#N)cc3)[C@H](c3cccc(F)c3)C[C@H](NC(=O)N3CCC3)[C@@]21O] 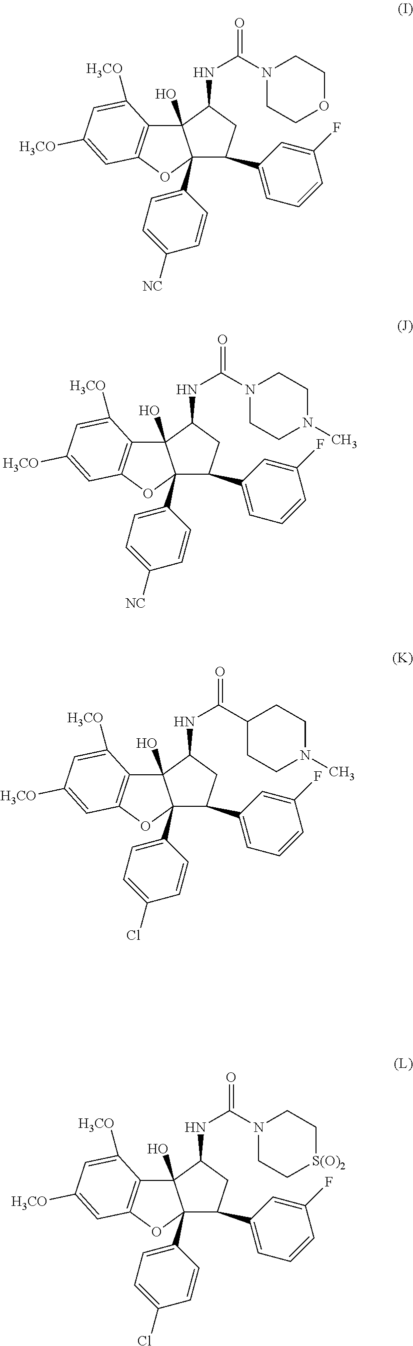 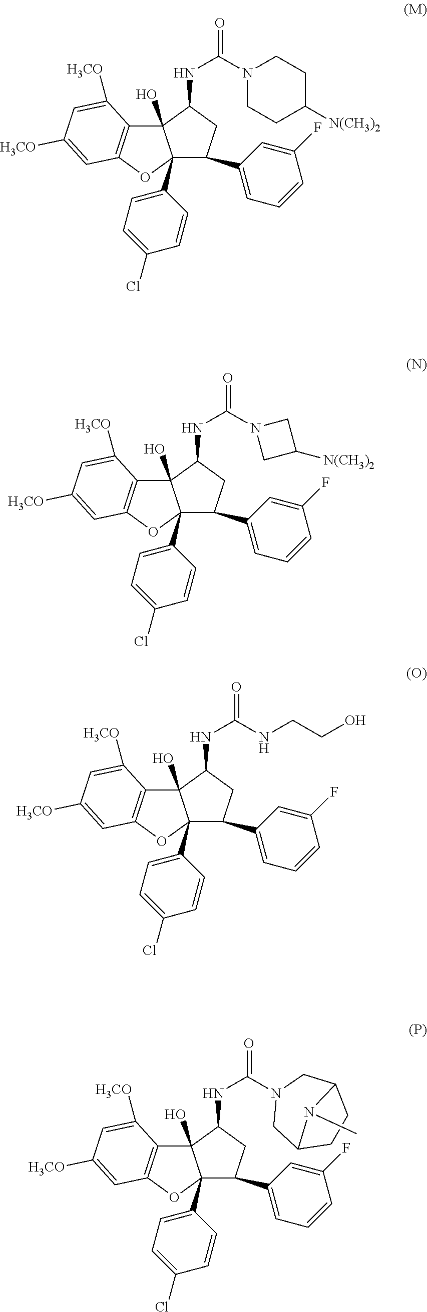 [SMILES: COc1cc(OC)c2c(c1)O[C@@]1(c3ccc(Cl)cc3)[C@H](c3cccc(F)c3)C[C@H](NC(=O)N3CCC(N(C)C)CC3)[C@@]21O ; COc1cc(OC)c2c(c1)O[C@@]1(c3ccc(Cl)cc3)[C@H](c3cccc(F)c3)C[C@H](NC(=O)N3CC(N(C)C)C3)[C@@]21O ; COc1cc(OC)c2c(c1)O[C@@]1(c3ccc(Cl)cc3)[C@H](c3cccc(F)c3)C[C@H](NC(=O)NCCO)[C@@]21O ; COc1cc(OC)c2c(c1)O[C@@]1(c3ccc(Cl)cc3)[C@H](c3cccc(F)c3)C[C@H](NC(=O)N3CC4CCC(C3)N4C)[C@@]21O] 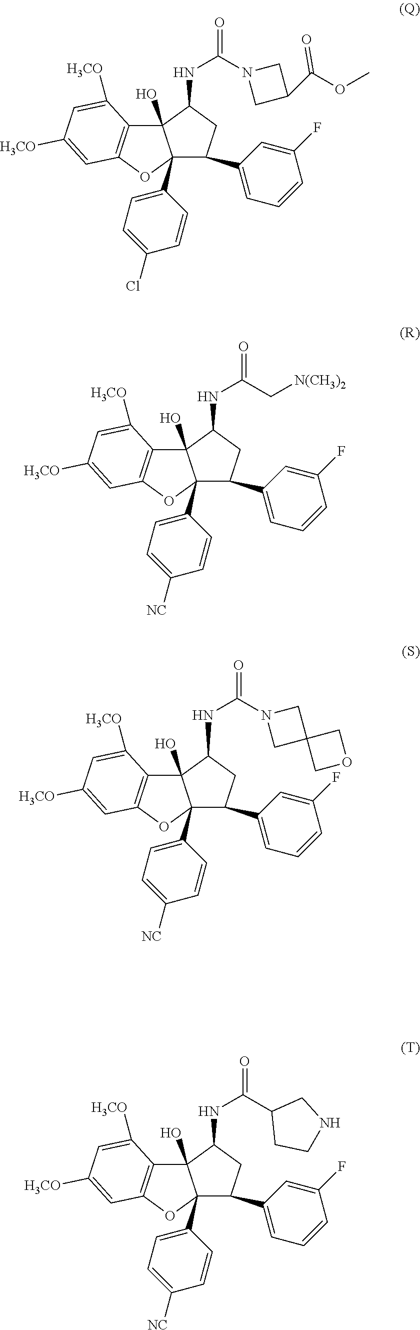 [SMILES: COC(=O)C1CN(C(=O)N[C@H]2C[C@@H](c3cccc(F)c3)[C@]3(c4ccc(Cl)cc4)Oc4cc(OC)cc(OC)c4[C@]23O)C1 ; COc1cc(OC)c2c(c1)O[C@@]1(c3ccc(C#N)cc3)[C@H](c3cccc(F)c3)C[C@H](NC(=O)CN(C)C)[C@@]21O ; COc1cc(OC)c2c(c1)O[C@@]1(c3ccc(C#N)cc3)[C@H](c3cccc(F)c3)C[C@H](NC(=O)N3CC4(COC4)C3)[C@@]21O ; COc1cc(OC)c2c(c1)O[C@@]1(c3ccc(C#N)cc3)[C@H](c3cccc(F)c3)C[C@H](NC(=O)C3CCNC3)[C@@]21O] 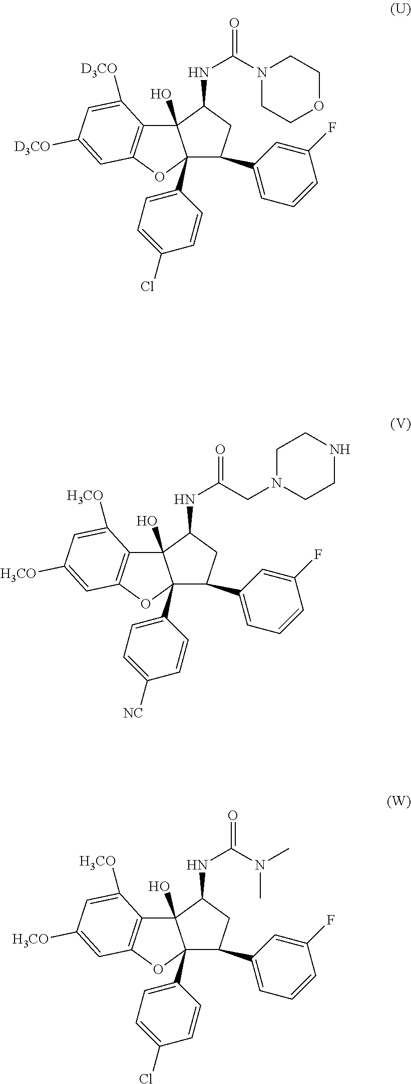
or mixtures selected from two or more compounds (A) to (W) and enantiomers thereof.

9. The process of claim 1, wherein the (+) ssRNA virus is a member of a family selected from togaviridae, flaviviridae, coronaviridae and retroviridae.

10. The process of claim 1, wherein the (−) ssRNA virus is a member of a family arenaviridae, bornaviridae, bunyviridae, filoviridae, othymyxoviridae, paramyxoviridae pneumoviridae or rhabdoviridae.

11. The process of claim 10, wherein from the virus is Dengue virus, Japanese encephalitis virus, Kyasanur Forest disease virus, Powassan virus, West Nile virus, Yellow fever virus, or Zika virus.

12. The process of claim 1, wherein from the virus is SARS-CoV, SARS-COV-2, MERS-CoV, HCOV-229E, HCoV-NL63, HCovC43 or HKU1.

13. The process of claim 1, wherein from the virus is HIV-1 and HIV-2.

14. The process of claim 1, wherein from the virus is Bundibugyo ebolavirus, Reston ebolavirus, Sudan ebolavirus, Taï Forest ebolavirus, Zaire ebolavirus or Bombali ebolavirus.

15. The process of claim 1, wherein the disease is dengue fever, MERS, COVID-19, SARS, ebobla fever, AIDS or zika fever.

16. The process of claim 1, wherein the disease comprises lung inflammation, respiratory distress, pulmonary fibrosis, pneumonia, cytokine storm, acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events rhabdomyolysis, neurocognitive deficits, and sensory deficits, or motor deficits.

17. A process of inhibiting virus activity in a subject in need thereof comprising administering to the subject a compound of formula (I) 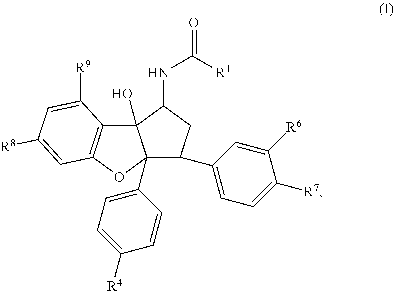
or a pharmaceutically acceptable salt thereof, wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl. C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-tr or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy, C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR₅ₐR^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen. C₁-C₄-alkyl and C₃-C₇ cycloalkyl:
R⁸ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁶ is selected from OCH₃, OCD₃, and
R⁹ is selected from OCH₃, OCD₃.

18. The process of claim 17, wherein the virus is a positive single-stranded RNA viruses (+) ssRNA) or negative single-stranded RNA virus ((−) ssRNA).

19. (canceled)

20. A pharmaceutical composition comprising at least one compound of formula (I) 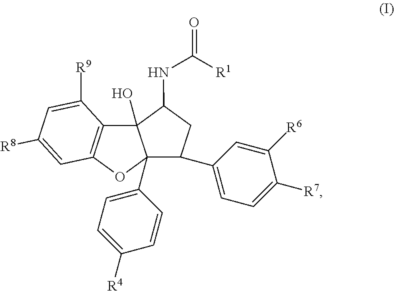
wherein
R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{a} and wherein the heterocyloalkyl is connected to the remaining molecule via a carbon atom;
NR²R³, wherein R² and R³ independently from each other are selected from hydrogen, C₁-C₄ alkyl, C₃-C₇-cycloalkyl and C₃-C₇ heterocyloalkyl, comprising 1, 2 or 3 identical of different heteroatoms or hetercatom-containing groups as ring members, selected from N. NR^{c}, O, S, SO and SO₂, and wherein the heterocyclalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicais R^{g};
R^{a} is selected from halogen, OH, C₃-C₇ cycloalkyl. C₁-C₃ alkoxy, phenyl, NR₅ₐR^{5b}, C₁-C₄-alkylsulfonyl, C₃-C₇ heterocyioalkyl, comprising 1, 2 or 3 identical or different heteroatoms or heteroatom-containing groups as ring members, selected from N, NR^{c}, O, S, SO, and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH:
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b}.
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁸ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃; and
R⁹ is selected from OCH₃, OCD₃, pharmaceutically acceptable salts thereof and a pharmaceutically acceptable diluent or carrier.


SEQUENCE LISTING (8 sequences)

SEQ ID NO: 1 - RNA, 96 nt, Dengue virus
  source 1..96: mol_type = mRNA
agttgttagtctacgtggaccgacaaagacagattctttgagggagctaagctcaacgtagttctaacagttttttaattagagagcagatctctg

SEQ ID NO: 2 - RNA, 469 nt, Ebola virus
  source 1..469: mol_type = mRNA
cggacacacaaaaagaaagaagaatttttaggatcttttgtgtgcgaataactatgaggaagattaataattttcctctcattgaaatttatatcggaatttaaattgaaattgttactgtaatcacacctggtttgtttcagagccacatcacaaagatagagaacaacctaggtctccgaagggagcaagggcatcagtgtgctcagttgaaaatcccttgtcaacacctaggtcttatcacatcacaagttccacctcagactctgcagggtgatccaacaaccttaatagaaacattattgttaaaggacagcattagttcacagtcaaacaagcaagattgagaattaaccttggttttgaacttgaacacttaggggattgaagattcaacaaccctaaagcttggggtaaaacattggaaatagttaaaagacaaattgctcggaatcacaaaattccgagt

SEQ ID NO: 3 - RNA, 292 nt, HCov 2229E virus
  source 1..292: mol_type = mRNA
acttaagtaccttatctatctacagatagaaaagttgctttttagactttgtgtctacttttctcaactaaacgaaatttttgctatggccggcatctttgatgctggagtcgtagtgtaattgaaatttcatttgggttgcaacagtttggaagcaagtgctgtgtgtcctagtctaagggtttcgtgttccgtcacgagattccattctacaaacgccttactcgaggttccgtctcgtgtttgtgtggaagcaaagttctgtctttgtggaaaccagtaactgttccta

SEQ ID NO: 4 - RNA, 335 nt, HI virus
  source 1..335: mol_type = mRNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagag

SEQ ID NO: 5 - RNA, 278 nt, MERS Co virus
  source 1..278: mol_type = mRNA
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatc

SEQ ID NO: 6 - RNA, 264 nt, SARS Co virus
  source 1..264: mol_type = mRNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaag

SEQ ID NO: 7 - RNA, 265 nt, SARS Co virus 2
  source 1..265: mol_type = mRNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 8 - RNA, 106 nt, Zika virus
  source 1..106: mol_type = mRNA
agttgttgatctgtgtgagtcagactgcgacagttcgagtctgaagcgagagctaacaacagtatcaacaggtttaatttggatttggaaacgagagtttctggtc